SEQUENCE LISTING (39 sequences)

SEQ ID NO: 1 - DNA (genomic), 1512 bp; double-stranded, linear
  CDS 48..1040
  sig_peptide 48..107
cccaaaggtaaagacactcaaggacagacatttttggcagagcatagatgaaaatggcaagttccctggctttccttctgctcaactttcatgtctccctcctcttggtccagctgctcactccttgctcagctcagttttctgtgcttggaccctctgggcccatcctggccatggtgggtgaagacgctgatctgccctgtcacctgttcccgaccatgagtgcagagaccatggagctgaagtgggtaagttccagcctaaggcaggtggtgaacgtgtatgcagatggaaaggaagtggaagacaggcagagtgcaccgtatcgagggagaacttcgattctgcgggatggcatcactgcagggaaggctgctctccgaatacacaacgtcacagcctctgacagtggaaagtacttgtgttatttccaagatggtgacttctatgaaaaagccctggtggagctgaaggttgcagcactgggttctaatcttcacgtcgaagtgaagggttatgaggatggagggatccatctggagtgcaggtccaccggctggtacccccaaccccaaatacagtggagcaacgccaagggagagaacatcccagctgtggaagcacctgtggttgcagatggagtgggcctatatgaagtagcagcatctgtgatcatgagaggcggctccggggagggtgtatcctgcatcatcagaaattccctcctcggcctggaaaagacagccagcatttccatcgcagaccccttcttcaggagcgcccagccctggatcgcagccctggcagggaccctgcctatcttgctgctgcttctcgccggagccagttacttcttgtggagacaacagaaggaaataactgctctgtccagtgagatagaaagtgagcaagagatgaaagaaatgggatatgctgcaacagagcgggaaataagcctaagagagagcctccaggaggaactcaagaggaaaaaaatccagtacttgactcgtggagaggagtcttccgtccgataccaataagtcagcctgatgctctaatggaaaaatggccctcttcwagcctggtgaggaaatgcttcagatgaggctccaccttggttaaataaattggatgtatggaaaaatagactgcagaaaaggggaactcatttagctcncgagtggtcgagtgaagattgaaaattaacctctgagggccagcacagcagctcatgcctgtaatcctagcactttgggaaggcttgaggagggcggrtcacaaggtcaggaggatcaaagaccatcctggctaacacggtggaaaccccgnctctactaaaaatacaaaaaataaaaaattagccgggncatggtgacgggcacctgtaggtcccagctactcgggaggctgaggcaggaggaatggcatgaacccggaaggcagrgcttgcagkgagccgagnatcaacgscactgcactccagcctgggaggacaagagcgaagactct

SEQ ID NO: 2 - protein, 331 aa
MKMASSLAFLLLNFHVSLLLVQLLTPCSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSNLHVEVKGYEDGGIHLECRSTGWYPQPQIQWSNAKGENIPAVEAPVVADGVGLYEVAASVIMRGGSGEGVSCIIRNSLLGLEKTASISIADPFFRSAQPWIAALAGTLPILLLLLAGASYFLWRQQKEITALSSEIESEQEMKEMGYAATEREISLRESLQEELKRKKIQYLTRGEESSVRYQ

SEQ ID NO: 3 - DNA (cDNA), 30 bp; single-stranded, linear
ggaagatctctccttgctcagctcagtttt

SEQ ID NO: 4 - DNA (cDNA), 34 bp; single-stranded, linear
gcgcagatctctagggctgggcgctcctgaagaa

SEQ ID NO: 5 - DNA (cDNA), 30 bp; single-stranded, linear
ggaagatctttattggtatcggacggaaga

SEQ ID NO: 6 - DNA (cDNA), 39 bp; single-stranded, linear
gcgcagatctccgccatcatgaaaatggcaagttccctg

SEQ ID NO: 7 - protein, 223 aa
MKMASSLAFLLLNFGVSLLLVQLLTPCSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSNLHVEVKGYEDGGIHLECRSTGWYPQPQIQWSNAKGENIPAVEAPVVADGVGLYEAVAASVIMRGGSGEGVSCII

SEQ ID NO: 8 - protein, 26 aa
RNSLLGLEKTASISIADPFFRSAQPW

SEQ ID NO: 9 - DNA (cDNA), 3974 bp; both-stranded, both
ggtacctaagtgagtagggcgtccgatcgacggacgccttttttttgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcgtcgacaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgtcgaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagcttaaaaaactgcaaaaaatagtttgacttgtgagcggataacaattaagatgtacccaattgtgagcggataacaatttcacacattaaagaggagaaattacatatg

SEQ ID NO: 10 - DNA (cDNA), 112 bp; both-stranded, both
aagcttaaaaaactgcaaaaaatagtttgacttgtgagcggataacaattaagatgtacccaattgtgagcggataacaatttcacacattaaagaggagaaattacatatg

SEQ ID NO: 11 - DNA (cDNA), 336 bp; both-stranded, both
caaatacagtggagcaacgccaagggagagaacatcccagctgtggaagcacctgtggttgcagatggagtgggcctatatgaagtagcagcatctgtnatcatgagaggcggctccggggagggtgtatcctgcatcatcagaaattccctcctcggcctggaaaagacagccagcatttccatcgcagaccccttcttcaggagcgcccagccctggttcgcagccctggcagggaccctgcctatnttgctgctgcttctcgccggagccagttacttcttgtggagacaacagaaggaaataactgctcttgtccagtgaagattagaaagt

SEQ ID NO: 12 - DNA (cDNA), 294 bp; both-stranded, both
ggtgaacgtntatgcagatggaaaggaagtggaagacaggcagagtgcaccgtatcgagggagaacttcgattctccgggatggcatcactgcagggaaggctgctctccgaatacacaacgtcacagcctctgacagtggaaagtacttgttttatttccaagatggtgacttctatgaaaaanccctggtggagctgaaggttgcagcactgggttctgatcttcacgttgatgtgaagggttacaaggatggagggatccatctggagtgcaaggtccactggctggtacc

SEQ ID NO: 13 - DNA (cDNA), 502 bp; both-stranded, both
taattcggcanaggttttccatactggaacccaaaggtaaagacactcaaggacagacatttttggcagagcatagatgaaaatggcaagttccctggctttccttctgctcaactttcatgtctccctcctcttggtccagctgctcactccttgctcagcttcagttttctgtgcttggaccctctgggcccatcctggccatggtgggtgaagacgctgatctgccctgtnacctgttcccgaccatggagtncagagaccatgggagcttgaagtgggtaaagttncagcctaaggcaggtggttgaacgtgttatgcagatgggaaaggaagttggaagacaggcagagttgcaccgttttcgaggggggaatttggatttnttcggggtgggcntcaatnncagggaaggntnnttttncgattacaaaaggtnaaaactttttacaatggaaagnatttgnnttattttncnagnnggggacttttttttnaaaa

SEQ ID NO: 14 - DNA (cDNA), 447 bp; both-stranded, both
gaattcggcanaggnttttccatactggaacccaaaggtaaagacactcaaggacagacatttttggcagagcatagatgaaaatggcaagttccctggctttccttctgntcaactttcatgtctccctcctcttggtccagctgctcactccttgctcagctcagttttctgtgcttggaccctctgggcccatcctggccatggtgggtgaagacgctgatctgccctgtcacctgttcccgaccatggagtgcagagaccatgggagctgaagtgggtaaagttccagcctaaggcaggtggttgaacgtgttatncagatgggaaagggaagttgggaagacaggcaagagtgncancnttattcganggggngnactttcgattttttnnggggntgggcattcaantnccnagggaaagggttgtttttccgnattacaa

SEQ ID NO: 15 - DNA (cDNA), 498 bp; both-stranded, both
aattcggcacgaggttttccatactggaacccaaaggtaaagacactcaaggacagacatttttggcagagcatagatgaaaatggcaagttccctggctttccttctgctcaactttcatgtctccctcctcttggtccagctgctcactccttgctcagctcagttttctgtgcttggaccctctgggcccatcctggccatggtgggtgaagacgctgatctgccctgtcacctgttcccgaccatgagttgcagagaccatgggagctngaagtgggtaaagttccagcctaaggcangtggtggaacgtgtnntncagatgggaaagngaagttgggaagaccaggcagagtnggcaccnttatttngagggnagggaatttnnggntttctnnggggnttgggcntcaatnggnnaggggnanggttgntttttnccggnttnncaaaaaagttnaacnnnctttnnagcaattgggaaaggactngnntta

SEQ ID NO: 16 - DNA (cDNA), 397 bp; both-stranded, both
aattcggcagaggttttccatactggaacccaaaggtaaagacactcaaggacagacatttttggcagagcatagatgaaaatggcaagttccctggctttccttctgctcaactttcatgtctccctcctcttggtccagctgctcactccttgctcagctcagttttctgtgcttgggaccctctgggcccatcctggccatggtgggtgaagacgctgatctgccctgtnacctgttcccgaccatgagtgcagagaccatggagctgaaagtgggtaagtttccagnctnaaggcaggtggtgaaacgtgtattncannttggnaaaggaagttggnaagaaagggnnggtngccacnttttngggggggggnaatttggntttttggggggt

SEQ ID NO: 17 - DNA (cDNA), 499 bp; both-stranded, both
aattcggcanaggnttttccatactggaacccaaaggtaaagacactcaaggacagacatttttggcagagcatagatgaaaatggcaagttccctggctttccttctgctcaactttcatgtctccctcctcttggtccagctgctcactccttgctcagctcagttttctgtgcttgggaccctctgggcccatcctggccatggtgggtgnagacgctgatctgccctgtcacctgttcccgaccatgnagtncagagaccatgggagctggaagtgggttaagttccagcctnaaggcaggtggtgaacgttttatgcagatgggaaagggaagttggaagacaggcagagtgccaacnttatnggagggnagaactttggntttttgcnggnntgggcatcaatnncaagggaagggntntttttccggataacaaaaagtnaaangnctttnnacaatggggnaattantggttttattttncaagatgggtg

SEQ ID NO: 18 - DNA (cDNA), 498 bp; both-stranded, both
aattcggcanaggtttttccatactggaacccaaaggtaaagacactcaaggacagacatttttggcagagcatagatgaaaatggcaagttccctggctttccttctgctcaactttcatgtctccctcctcttggtccagctgctcactccttgctcagctcagttttctgtgcttngaccctctgggcccatcctggccatggtgggtgaagacgctgatcttgccctgtnacctgttcccgaccatgagtncagagaccatgggaggctgnaagtggggtaagttccagccttaaggcangtnggtgnaacgtgttatgcagatgggnaagggaagtnggaaggacanggcanaagttgcancntttttgnggggngaactttcggttttttgcgggatggggattcaatnncagggaaaggttgtttttncngaatncanaaagttnanaagcttttgacaaatnggaagttacttgtngttantttccaaga

SEQ ID NO: 19 - DNA (cDNA), 482 bp; both-stranded, both
aattcggcanaggattttccatactggaacccaaaggtaaagacactcaaggacagacatttttggcagagcatagatgaaaatggcaagttccctggctttccttctgctcaactttcatgtctccctcctcttggtccagctgctcactccttgctcagctcagttttctgtgcttggtaccctctgggcccatcctggccatggtgggtgaagacgctgatctgccctgtnnacctgttncccgnaccatggagtgcaggagaaccatggagctgnaagtggggtaaagttcccagcctaaaggcaggtggtngaacgtgttattgcagatggtaaaggnaagttggnaggacaggncagagntgcaaccnttttcgggggggggaattnnngatttttggggggggttggncattcaattngnagggnaagggttngttntggcggaatnagaanaagngtgaaaaagtctttttgaa

SEQ ID NO: 20 - DNA (cDNA), 447 bp; both-stranded, both
aattcggcanaggttttccatactggaacccaaaggtaaagacactcaaggacagacatttttggcagagcatagatgaaaatggcaagttccctggctttccttctgctcaactttcatgtctccctcctcttggtccagctgctcactccttgctcagctcagttttctgtgcttgggaccctctgggnccatcctggccatggtgggtgtagnacgctggatctgccctgtcanctgtttcccgaccatgagtgcaggggaccatgggagctggaagtggggtaaagtttccagccttaagggcaggttnggtggaacgtggttattgcangatggggaaaaggtagtnngnaggacangggcagaagtggncaccgttattcgtgggggaggaacttttngatttttgcgggggnnnggcatcaattttcaggggnaagggtt

SEQ ID NO: 21 - DNA (cDNA), 322 bp; both-stranded, both
gaattcggcanagggttttccatactggaacccaaaggtaaagacactcaaggacagacatttttggcagagcatagatgaaaatggcaagttccctggctttccttctgntcaactttcatgtctccctcctcttggtccanctgctcantccttgctcanctcagttttctgtgncttgggaccctctgggcccatnctggccatggtgggttnnagacgctgattctgccctgtnnanctgttcccggaccatgagttncanagaccatgggaggctttaagtggggtnaantttccanccttaagggcaaagttnggt

SEQ ID NO: 22 - DNA (cDNA), 301 bp; both-stranded, both
gagatggagtcttgctgtctcccaggctggagtgcagtggcgcaatctcagctcactgcaagctccgcctcccgggttcatgccattctcctgcctcagcctcccgagtagctgggactacaggtgcctgccaccacgcccggccaatttttcgtatttttagtagagatgggatttcaccgtgttagccaggatggtctcgatctcctgacctcgtgatctgcccgnctcggcctcccaaagtgccgggattacaggcatgagccaccacacccggcgaccccagttacttctttagtta

SEQ ID NO: 23 - DNA (cDNA), 445 bp; both-stranded, both
aattcggcacaggnaaaaatagactgcagaaaaggggnactcatttagctcacgagtggtcgagtgaagattgaaaattaacctctgagggccagcacagcagctcatgcctgtaatcctagcactttgggaaggctgaggagggcggatcacaagcctgatttttcctgcatgggaagagcccacatgnngccctgaggttcccttcccagggncagntccaggatcgagatgactgtgagtggttgtggagttaagaccctatggactncttcccagttggtttntcagagctntagacccagcattcntgggtttggttttgcagagtnttttngttgngagnattaagttngnatttcccacaggggatttgganttttaaagggattagggggccaaatttngtttaattaatgggggnaaaantttttttcccacccaa

SEQ ID NO: 24 - DNA (cDNA), 468 bp; both-stranded, both
gaactattaactgccttttcttcttgtgggctgtgattttcagaggggaatgctaagagtatctccgtgatatgcagcatgaatgaaaatggcaagtttcctggccttccttctgctcaactttcgtgtctgcctccttttgcttcagctgctcatgcctcactcagctcagttttctgtgcttggaccctctggcccatcctggccatggtgggtgaagacgctgatctgccctgtcacctgttcccgaccatgagtgcagagaccatggagctgaagtgggtaagttccagcctaaggcaggtggtgaacgtgtatgcagatggaaaggaagtggaagacaggcagagtgcaccgtattcgagggagaacttcgattctgcgggatggcatcactgcaggggaaggctgctttccgaatacacaacgtcacagcctctgacagtnggaaagtacctgtgttatttt

SEQ ID NO: 25 - DNA (cDNA), 336 bp; both-stranded, both; identical to SEQ ID NO: 11

SEQ ID NO: 26 - DNA (cDNA), 440 bp; both-stranded, both
ntcntgacttctccaantgggaataccaangggattggttttccatacttggaacccaaaggtaaagacactcaaggacagacatttttggcaganagtagatgaaaatggcaagttccctggctttccttctgctcaactttcatgtctccctcctcttggtccagctgctcactccttgctcagctcagttttctgtgcttggaccctctggcccatcctggccatggtgggtgaagacgctgatctgccctgtcacctgttcccgaccatgagtgcagagaccatggagctgaagtgggtaagttccagcctaaaggcaggtggtgaacgtgtatgcagatggaaaggaagtgggaagacaggcagagtgcaccgtatcgaggggagaaactttcgatttctgacggggatggcatcactgcaggaaaggctgctct

SEQ ID NO: 27 - DNA (cDNA), 444 bp; both-stranded, both
nttcggcacggagaactattaactgcctttcttctgtgggctgtgattttcagaggggaatgctaagagtatctcctgatatgcagcatgaatgaaaatggcaagtttcctggccttccttctgctcaactttcgtgtctgcctccttttgcttcagctgctcatgcctcactcagctcagttttctgtgcttggaccctctgggcccatcctggccatggtgggtgaagacgctgatctgccctgtcacctgttcccgaccatgagtgcagagaccatggagctgaagtgggtaagttccagcctaaggaggtggtgaacgtgtatgcagatggaaaggaagtggaagacaggcagagtgcaccgtatcgagggagaacttcgattctgcgggatggcattcactgcagggaaggctgcttttccgattacacaactcacagn

SEQ ID NO: 28 - DNA (cDNA), 294 bp; both-stranded, both; identical to SEQ ID NO: 12

SEQ ID NO: 29 - DNA (cDNA), 390 bp; both-stranded, both
agaactattaactncctttcttctntgggctgtgattttcagaggggaatgctaagagtatctcctgatatgcagcatgaatgaaaatggcaagtttcctggccttccttctgctcaactttcgtgtctgcctccttttgcttcagctgctcatgcctcactcagctcagttttctgtgcttggaccctctgggcccatcctggccatggtnggtgaagacgctgatctnccctgtcacctgttcccgaccatgagtncagagaccatggagctgaagtgggtaagttccagccnaaggcaggatggtgaacgtntatgcagatggaaaggaagtggaagacaggcagagtgcaccntattcgagggagaacttngattctggcggggat

SEQ ID NO: 30 - DNA (cDNA), 336 bp; both-stranded, both
gttttccatactggaacccaaaggtaaagacactcaaggacagacatttttggcagagcactagatgaaaatggcaagttccctggctttccttctgctcaactttcatgtctccctcctcttggtccagctgctcactccttgctcagctcagttttctgtgcttggaccctctggcccatcctggccatggtgggtgaagacgctgatctgccctgtcacctgttcccgaccatgagtgcagagaccatggagcttgaagtgggtaagttccagcctaagnagggtggtgaacggtggtatgcagattggaaaangaagtggaagacanggcag

SEQ ID NO: 31 - DNA (cDNA), 441 bp; both-stranded, both
gaactattaactgcctttcttctgtgggctgtgattttcagaggggaatgctaagagtatctcctgatatgcagcatgaatgaaaatggcaagtttcctggccttccttctgctcaactttcgtgtctgcctccttttgcttcagctgctcatgcctcactcagctcagttttctgtgcttggaccctctgggcccatcctggccatggtgggtgaagacgctgatctgccctgtcacctgttcccgaccatgagtgcagagaccatggagctgaagtgggtaagttccagcctaaggcaggtggtgaacgtgtatgcagatggaaaggaagtgggaagacagggcagagtgcaccgtattcgagggagaaactttcgattnttgcggggatgggcatcactgncaggggaagggttgcttttccgaattacacaacgttc

SEQ ID NO: 32 - DNA (cDNA), 435 bp; both-stranded, both
gctcgacttcctgctgtaccactcaggaattctttctaaagaaagaatgtgtctttcttaagggttgcagagaagctaagttgggaggcagtgcagacaattgctagtgagcagccaggagtgtctgcagtacttttggaagagggactctgcatctgctctagatcctacagagaagtgttctcagaacaaaacctggaggctcacctgcaaccttcagctccaccagggctttttcgtagaagtcaccatcttggaaataacacaagtactttccactgtcagaggctgtgacgttgtgtaattcggagagcagccttccctgcagtgatgccatcccgcagaatcgaagttctccctcgatacggtgcactctgcctgtcttccacttcctttccatctgcatacacgttcaccacctgccttaggctggaa

SEQ ID NO: 33 - DNA (cDNA), 413 bp; both-stranded, both
tctgattctccaatgggaataccaagggatggttttccatactggaacccaaaggtaaagacactcaaggacagacatttttggcagagatagatgaaaatggcaagttccctggctttccttctgctcaactttcatgtctccctcctcttggtccagctgctcactccttgctcagctcagttttctgtgcttggaccctctgggcccatcctgggccatggtgggtgaagacgctgatctgccctgtcacctgttcccgaccatggagtgcagagaccatgggagctggaagtggggtaaagtttccaggcctaaaggcagggtgggtgaacgtgttatggcagatgggaaagggaagtgggaagaacagggcagagttgcaccntttttcnaggggagaaatttcgatt

SEQ ID NO: 34 - DNA (cDNA), 207 bp; both-stranded, both
tctaatcttcacgtcgaagtgaagggttatgaggatggagggatccatctggagtgcaggtccaccggctggtacccccaaccccaaatacagtggagcaacgccaagggagagaacatcccagcttgtggaagcacctgtggttgcagatggagtgggcctatatgaagtagcagcatctgtgatcatgagaggcggctcccgggg

SEQ ID NO: 35 - DNA (cDNA), 445 bp; both-stranded, both
ctgtctcccaggctggagtgcagtggcgtgatctcggctcactgcaagctctgccttccgggttcatgccattctcctgcctcagcctcccgagtagctgggactacaggtgcccgtcaccatgcccggctaattttttattttttgtatttttagtagagacggggtttcaccgtgttagccaggatggtcttgatctcctgaccttgtgatccgccctcctcagccttccaaagtgctaggattacaggcatgagctgctgtgctggccctcagaggttaattttcaatcttcactcgaccactccgtgaagctaaatgaagttcccccttttctgcaagtctaatttttcccatacatcccaatttnaatttaacaaaggtggaagcctcaatctgaaagcattttcctcaacaaggcntgaaagaggggcaatttttccan

SEQ ID NO: 36 - DNA (cDNA), 349 bp; both-stranded, both
gaactattaactgcctttcttctgtgggctgtgattttcagaggggaatgctaagagtatctcctgatatgcagcatgaatgaaaatggcaagtttcctggccttccttctgctcaactttcgtgtctgcctccttttgcttcagctgctcatgcctcactcagctcagttttctgtgcttggaccctctgggcccatcctggccatggtgggtgaagacgctgatctgccctgtcacctgttcccgaccatgagtgcagagaccatgggagctgaagtgggtaagttccagccttaaggcaggtggtnaacgtgtattgcagatgggaaangaagttggaagacaggc

SEQ ID NO: 37 - DNA (cDNA), 388 bp; both-stranded, both
gaactattaactgcctttcttctgtgggctgtgattttcagaggggaatgctaagagtatctcctgatatgcagcatgaatgaaaatggcaagtttcctggccttccttctgctcaactttcgtgtctgcctccttttgcttcagctgctcatgcctcactcagctcagttttctgtgcttggaccctctgggcccatcctggccatggtgggtgaagacgctgatctgccctgtcacctgttcccgaccatgagtgcagagaccatgggagctgaagtggggtaagttccagcctaaggcaggtgggtgaacgtgtatggcagatgggaaagggaagtgggaaggacagggcagagtggcaccgtattcgaggggagaactttcgat

SEQ ID NO: 38 - DNA (cDNA), 420 bp; both-stranded, both
ttttcattcatccattttatttaacaaagtggacgcctcatctgaagcatttcctcaccaggcttgaagagggcctttttccattcattataggctgaatgtctctctccccgagatgcatactgganacttctccatctgagttcctccaggagcttcactcttgtgctttgttcttgcttcattgtgctccatgccatttctctnaactcttgctctctctttttctttctgaactgagtcttttttnccncctgctgttgccacaggaagnaaccggctcccccaagaagcagcagcaagacaggcagggttccctgcagggggcgggcgantccacctctggggcgntcctgaagaagggggtctncgatggaaatgctgggctgttttttcaggggccagggaggggaacttcgg

SEQ ID NO: 39 - DNA (cDNA), 393 bp; both-stranded, both
gaactattaactgcctttcttctgtgggctgtgattttcagaggggaatgctaagagtatctcctgatatgcagcatgaatgaaaatggcaagtttcctggccttccttctgctcaactttcgtgtctgcctccttttgcttcagctgctcatgcctcactcagctcagttttctgtgcttgggaccctctgggcccatcctgggccatgggtgggtgaagacgctgatctgccctgtcacctgttcccgaccatgagtgcagagaccatgggagctgaagtggggtaagttccagcctaaggcagggtgggtgaacgtgtattgcagatgggaaagggaagttggaaggacagggcagagttncaccntattcgaggggagaactttcgatt

(19) United States
(12) Patent Application Publication
(10) Pub. No.: US 2003/0166898 A1
(43) Pub. Date: Sep. 4, 2003

(54) Myelin oligodendrocyte glycoprotein-like protein (MOGp)

(75) Inventors: Arvind Chopra, Gaithersburg, MD (US); Henrik S. Olsen, Gaithersburg, MD (US); Reiner L. Gentz, Rockville, MD (US); Steven M. Ruben, Olney, MD (US)

Correspondence Address: HUMAN GENOME SCIENCES INC, 9410 KEY WEST AVENUE, ROCKVILLE, MD, 20850

(73) Assignee: Human Genome Sciences, Inc.

(21) Appl. No.: 10/197,844

(22) Filed: Jul. 19, 2002

Related U.S. Application Data
(63) Continuation of application No. 09/015,963, filed on Jan. 30, 1998, now abandoned.
(60) Provisional application No. 60/035,445, filed on Jan. 30, 1997.

(51) Int. Cl. C07H 21/02; C07H 21/04

(52) U.S. Cl. 536/23.1

(57) ABSTRACT

The present invention relates to a novel MOGp protein which is a member of the Ig superfamily. In particular, isolated nucleic acid molecules are provided encoding the human MOGp protein. MOGp polypeptides are also provided as are vectors, host cells and recombinant methods for producing the same. Also provided are diagnostic and therapeutic methods for detecting and treating cancer, inflammation, and multiple sclerosis (MS).

### CROSS REFERENCE TO RELATED APPLICATIONS

This application claims the benefit of U.S. Provisional Application No. 60/035,445, which is incorporated herein by reference.

### BACKGROUND OF THE INVENTION

1. Field of the Invention

The present invention relates to a novel member of the immunoglobin gene superfamily. More specifically, isolated nucleic acid molecules are provided encoding a human myelin oligodendrocyte glycoprotein-like protein (MOGp). MOGp polypeptides are also provided, as are vectors, host cells and recombinant methods for producing the same. Also provided are diagnostic and therapeutic methods for detecting and treating cancer, inflammation, and multiple sclerosis (MS).

2. Related Art

The immunoglobin (Ig) gene superfamily is comprised of a diverse group of genes that share evolutionary homology. Members of this superfamily are often associated with immune recognition, cell adhesion, or cell surface interaction. These proteins are generally integral membrane proteins comprising one or more extracellular domains, a transmembrane region, and an intracellular domain. See Hunkapillar et al., Adv. Immunol. 44:1-63 (1989).

Several different members of the Ig gene superfamily are associated with cells of the nervous system. While the most prominent proteins of myelin are proteolipid proteins and myelin-basic proteins, one quantitatively minor myelin protein that is a member of the Ig gene superfamily is myelin-associated glycoprotein (MAG). See Stoffel, Angew. Chem. Int. Ed. Engl. 29: 958-976 (1990).

Another minor component of myelin that is a member of Ig gene superfamily is myelin oligodendrocyte glycoprotein (MOG). Linington et al., J. Neuroimmunol. 6:387-396 (1984). It is believed that MOG plays a key role in the completion or maintenance of the myelin sheath. See Matthieu et al., Dev. Neurosci. 12:293-302 (1990). Native MOG has been purified and characterized, and found to migrate primarily as a 25-28 kDa doublet in SDS-PAGE immunoblots. A minor 54 kDa dimer band is also observed. Amiguet et al., J. Neurochem. 58: 1676-1682 (1992).

Several MOG genes have been cloned and the nucleotide and deduced amino acid sequences have been determined. See Gardinier et al., J. Neurosci. Res. 33:177-187 (1992)(rat MOG); Pham-Dinh et al., Proc. Natl. Acad Sci (USA) 90:7990-7994 (1993)(bovine MOG); Hilton et al., J. Neurochem. 65:309-318 (1995)(human MOG).

MOG has been localized at the extracellular surface of myelin sheaths and oligodendrocytes. Brunner et al., J. Neurochem. 52:298-304 (1989). Moreover, MOG appears on the surface of oligodendrocytes during in vitro development 1-2 days after other oligodendrocyte markers. Scolding et al., J. Neuroimmunol. 22:169-176 (1989).

Multiple sclerosis (MS) is a disease of the CNS characterized by perivascular inflammation accompanied by primary demyelination. A predominant T-cell immune response directed to the MOG antigen has been observed in MS patients, providing evidence that an autoimmune response to MOG may be involved in the pathogenesis of MS. Kerlero de Rosbo et al., J. Clin. Invest. 92:2602-2608 (1993); Sun et al., J. Immunol. 146:1490-1495 (1991); Steinman, Proc. Natl. Acad. Sci. USA 90: 7912-7914; Kiao et al., J. Neuroimmunol. 31:91-96 (1991).

Other membrane proteins are associated with cancer tissue. For example, a splice variant of CD44 (also known as Pgp-1, Hermes-3, HCAM, and ECMRIII) has been shown to play a role in tumor cell metastasis. Guthert et al., Cell 65: 13-24 (1991). Moreover, CD33 monoclonal antibodies are useful in the immunodiagnosis of acute leukemias. Griffin, J. D. et al., Leuk Res. 8: 521 (1984).

It is believed that other members of the Ig superfamily may be associated with CNS function generally and myelin specifically or are associated with cancer tissue. Therefore, there is a need in the art to identify and characterize these proteins.

### SUMMARY OF THE INVENTION

The present invention provides isolated nucleic acid molecules recombinant vectors, and host cells comprising a polynucleotide encoding the MOGp polypeptide having the amino acid sequence is shown in FIG. 1 (SEQ ID NO:2) or the amino acid sequence encoded by the cDNA clone deposited in a bacterial host as ATCC Deposit Number 97709 on Sep. 10, 1996.

The present invention also relates to methods of making such vectors and host cells and for using them for the production of MOGp polypeptides or peptides by recombinant techniques.

The invention further provides an isolated MOGp polypeptide, antibodies specific for the MOGp polypeptide, and methods of isolating these antibodies.

This invention also provides a diagnostic method useful during diagnosis of a CNS disorder such as MS, which involves: (a) providing a biological sample from an individual to be tested for MS; (b) assaying the biological sample for the amount of antibody to MOGp; (c) comparing the amount of MOGp antibody in the biological sample to the amount of MOGp antibody in a standard sample from an individual not having MS; and (d) correlating an enhanced amount of the antibody in the biological sample relative to the standard with an increased probability MS.

An additional aspect of the invention is related to a method of treating MS or ameliorating MS symptoms comprising administering to an individual in need of treatment a composition comprising a therapeutically effective amount of a soluble fragment of MOGp in admixture with a pharmaceutically acceptable carrier. For example, MOGp protein or fragments can be used to antagonize the binding of autoantibodies associated with MS to MOG, thereby preventing demyelination associated with MS.

The invention further provides a diagnostic method useful for the diagnosis or prognosis of cancer comprising: (a) assaying MOGp expression level in cells or body fluids of an individual; and (b) comparing the MOGp expression level with a standard MOGp expression level, whereby an increase in the MOGp expression level compared to the standard expression level is indicative of an increased probability of cancer.

The invention also provides a diagnostic method useful further diagnosis or prognosis of inflammation comprising: (a) assaying MOGp expression levels in cells or body fluids of an individual; and (b) comparing the MOGp expression level with a standard MOGp expression level, whereby an increase in the MOGp expression level compared to the standard expression level is indicative of an increased probability of inflammation.

A further aspect of this invention related to a method of treating diseases associated with MOGp expression such as cancer or inflammation comprising administering a therapeutically effective amount of a soluble MOGp functional derivative or an anti-MOGp antibody.

### BRIEF DESCRIPTION OF THE FIGURES

FIG. 1 shows the nucleotide (SEQ ID NO:1) and deduced amino acid (SEQ ID NO:2) sequences of MOGp. The protein has a leader sequence of about 29 amino acid residues (underlined) and a deduced molecular weight of about 34 kDa. An alternative potential leader sequence is about 21 amino acid residues (residues 1-21) in length.

FIG. 2 shows a schematic representation of the pHE4-5 expression vector (SEQ ID NO:9) and the subcloned MOGp cDNA coding sequence. The locations of the kanamycin resistance marker gene, the MOGp coding sequence, the oriC sequence, and the lacIq coding sequence are indicated.

FIG. 3 shows the nucleotide sequence of the regulatory elements of the pHE promoter (SEQ ID NO:10). The two lac operator sequences, the Shine-Delgarno sequence (S/D), and the terminal HindIII and NdeI restriction sites (italicized) are indicated.

### DETAILED DESCRIPTION

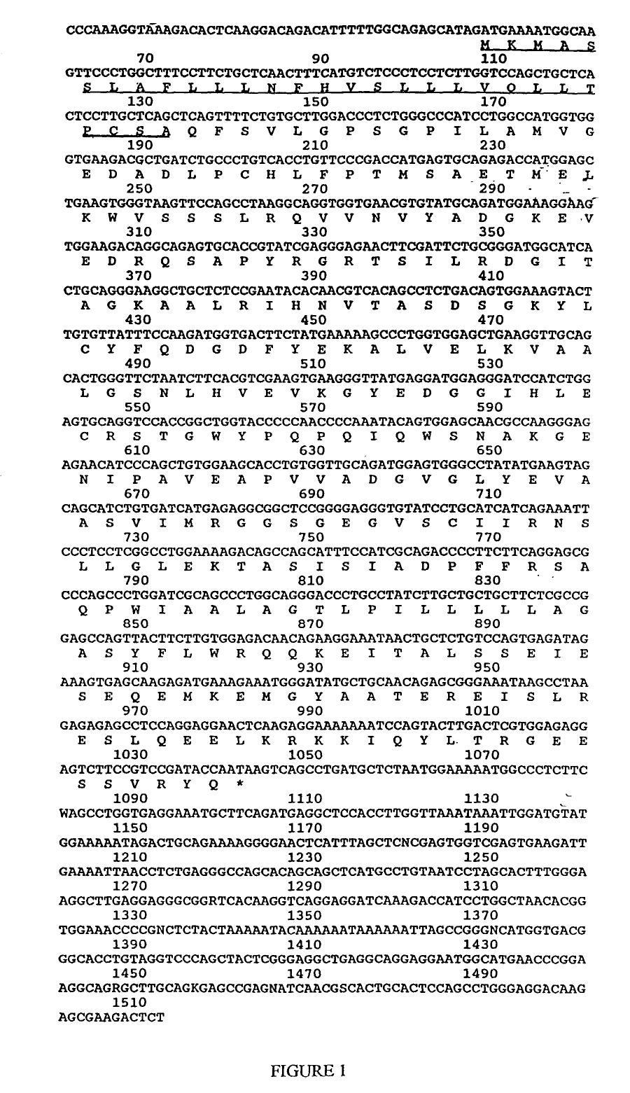

The present invention provides isolated nucleic acid molecules comprising a polynucleotide encoding a MOGp polypeptide having the amino acid sequence shown in FIG. 1 (SEQ ID NO:2), which was determined by sequencing a cloned cDNA. The MOGp protein of the present invention shares sequence homology with human myelin oligodendrocyte glycoprotein (MOG), whose sequence is disclosed in FIG. 1 of Hilton et al., supra. The nucleotide sequence shown in FIG. 1 (SEQ ID NO:1) was obtained by sequencing the HRDCD54 clone, which was deposited on Sep. 10, 1996 at the American Type Culture Collection, 12301 Park Lawn Drive, Rockville, Md. 20852, and given accession number 97709. The deposited clone is contained in the pBluescript SK(−) plasmid (Stratagene, La Jolla, Calif.).

Nucleic Acid Molecules

Unless otherwise indicated, all nucleotide sequences determined by sequencing a DNA molecule herein were determined using an automated DNA sequencer (such as the Model 373 from Applied Biosystems, Inc.), and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

Using the information provided herein, such as the nucleotide sequence in FIG. 1, a nucleic acid molecule of the present invention encoding a MOGp polypeptide may be obtained using standard cloning and screening procedures, such as those for cloning cDNAs using mRNA as starting material. Illustrative of the invention, the nucleic acid molecule described in FIG. 1 (SEQ ID NO:1) was discovered in a cDNA library derived from human rhabdomyosarcoma. The gene was also identified in cDNA libraries from lymphoid tissues such as spleen and peripheral blood lymphocytes, nasal poly, Raji cells, T-cell lymphomas, bone marrow, Hodgkins lymphoma, activated T-cells, activated epithelial cells, primary dendritic cells, DAM-1 cell line, cosinophils, fetal heart, 6 week embryo tissue, fetal liver, endometrial tumor, and placenta. These data indicate that this receptor is induced in activated or highly proliferating cells and that MOGp may be used as a tumor cell marker.

The determined nucleotide sequence of the MOGp cDNA of FIG. 1 (SEQ ID NO:1) contains an open reading frame encoding a protein of 331 amino acid residues, with an initiation codon at positions 49-51 of the nucleotide sequence in FIG. 1 (SEQ ID NO:1), a predicted leader sequence of about 29 amino acid residues, and a deduced molecular weight of about 34 kDa. The amino acid sequence of the predicted mature MOGp is shown in FIG. 1 (SEQ ID NO:2) from amino acid residue 30 to residue 331. An alternative leader sequence of about 21 amino acid residues is predicted. In the event of a leader sequence of about 21 amino acid residues, an alternative mature form of MOGp, from about amino acid residue 22 to residue 331 of FIG. 1 (SEQ ID NO:2), is obtained.

The MOGp protein shown in FIG. 1 (SEQ ID NO:2) is an immunoglobulin-like molecule and is about 33% identical and about 55% similar to human myelin oligodendrocyte glycoprotein. MOGp also has significant sequence homology to butyrophilin, a milk glycoprotein that is involved in the regulation of secretion during lactation.

As indicated, the present invention also provides the mature form(s) of the MOGp polypeptide of the present invention. According to the signal hypothesis, proteins secreted by mammalian cells have a signal or secretory leader sequence which is cleaved from the mature protein once export of the growing protein chain across the rough endoplasmic reticulum has been initiated. Most mammalian cells and even insect cells cleave secreted proteins with the same specificity. However, in some cases, cleavage of a secreted protein is not entirely uniform, which results in two or more mature species on the protein. Further, it has long been known that the cleavage specificity of a secreted protein is ultimately determined by the primary structure of the complete protein, that is, it is inherent in the amino acid sequence of the polypeptide. Therefore, the present invention provides a nucleotide sequence encoding the mature MOGp polypeptides having the amino acid sequence encoded by the cDNA clone contained in the host identified as ATCC Deposit No. 97709 and as shown in FIG. 1 (SEQ ID NO:2). By the mature MOGp protein having the amino acid sequence encoded by the cDNA clone contained in the host identified as ATCC Deposit No. 97709 is meant the mature form(s) of the MOGp protein produced by expression in a mammalian cell, e.g., COS cells, as described below, of the complete open reading frame encoded by the human DNA sequence of the clone contained in the vector in the deposited host. As indicated below, the mature MOGp protein having the amino acid sequence encoded by the cDNA clone contained in ATCC Deposit No. 97709 may or may not differ from the predicted “mature” MOGp protein shown in FIG. 1 (amino acids from about 30 to about 331) depending on the accuracy of the predicted cleavage site based on computer analysis.

Methods for predicting whether a protein has a secretory leader as well as the cleavage point for that leader sequence are available. For instance, the methods of McGeoch (Virus Res. 3:271-286 (1985)) and von Heinje (Nucleic Acids Res. 14:4683-4690 (1986)) can be used. The accuracy of predicting the cleavage points of known mammalian secretory proteins for each of these methods is in the range of 75-80%. von Heinje, supra. However, the two methods do not always produce the same predicted cleavage point(s) for a given protein.

In the present case, the predicted amino acid sequence of the complete MOGp polypeptide of the present invention were analyzed by a computer program (DNA Star), which is an expert system for predicting the cellular location of a protein based on the amino acid sequence. The hydrophobicity plot of MOGp, as shown by this program, predicted the cleavage sites between amino acids 25 and 26 in FIG. 1 (SEQ ID NO:2). Thereafter, the complete amino acid sequences were further analyzed by visual inspection, applying a simple form of the (−1,−3) rule of von Heinje. von Heinje, supra.

However, a cleavage site between residues 25 and 26 would result in the N-terminal amino acid of the mature protein being proline. It is not believed that proline is the N-terminal amino acid of MOGp since mature proteins typically do not have proline at their N-terminal amino acid. Moreover, it is also known that naturally produced mature MOGp protein has a blocked N-terminus. Proline typically cannot be modified to result in a blocked N-terminus. However, glutamine (Q) is often modified and is the most likely cause of the N-terminal block. Therefore, the site of cleavage is likely prior to position 22 or prior to position 30.

In order to distinguish between cleavage between positions 21 and 22, and between positions 29 and 30, the following considerations are helpful. First, the cleavage event typically requires an upstream alpha helix, which is seen in MOGp between position 14-21. Second, the alanine-glutamine junction at positions 29 and 30 is a good cleavage site. Third, the presence of proline at position −4 relative to the cleavage site is characteristic of a cleavage site. There is a proline at position 26 in MOGp. These criteria make it likely that the cleavage site is between residues 29 and 30, i.e., a 29 amino acid leader. However, for the reasons discussed supra, an alternative leader consisting of the first 21 amino acids of the MOGp protein is also predicted.

As one of ordinary skill would appreciate, due to the possibilities of sequencing errors discussed above, as well as the variability of cleavage sites for leaders in different known proteins, the actual MOGp polypeptide encoded by the deposited cDNA comprises about 331 amino acids, but may be anywhere in the range of 325-350 amino acids; and the actual leader sequence of this protein is predicted to be about 29 amino acids, but may be anywhere in the range of about 15-50 amino acids.

As indicated, nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

By “isolated” nucleic acid molecule(s) is intended a nucleic acid molecule, DNA or RNA, which has been removed from its native environment. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Isolated nucleic acid molecules of the present invention include DNA molecules comprising an open reading frame (ORF) shown in FIG. 1 (SEQ ID NO:1); DNA molecules comprising the coding sequence for the mature MOGp protein shown in FIG. 1 (last 310 amino acids) (SEQ ID NO:2); and DNA molecules which comprise a sequence substantially different from those described above but which, due to the degeneracy of the genetic code, still encode the MOGp protein. The genetic code is well known in the art. Thus, it would be routine for one skilled in the art to generate the degenerate variants described above.

In another aspect, the invention provides isolated nucleic acid molecules encoding the MOGp polypeptide having an amino acid sequence encoded by the cDNA clone contained in the plasmid deposited as ATCC Deposit No. 97709. Preferably, this nucleic acid molecule will encode the mature polypeptide encoded by the above-described deposited cDNA clone. The invention further provides an isolated nucleic acid molecule having the nucleotide sequence shown in FIG. 1 (SEQ ID NO:1) or the nucleotide sequence of the MOGp cDNA contained in the above-described deposited clone, or a nucleic acid molecule having a sequence complementary to one of the above sequences. Such isolated molecules, particularly DNA molecules, are useful as probes for gene mapping, for in situ hybridization with chromosomes, and for detecting expression of the MOGp gene in human tissue, for instance, by Northern blot analysis.

The present invention is further directed to fragments of the isolated nucleic acid molecules described herein. By a fragment of an isolated nucleic acid molecule having the nucleotide sequence of the deposited cDNA or the nucleotide sequence shown in FIG. 1 (SEQ ID NO:1) is intended fragments at least about 15 nucleotides, and more preferably at least about 20 nucleotides, still more preferably at least about 30 nucleotides, and even more preferably, at least about 40 nucleotides in length which are useful as diagnostic probes and primers as discussed herein. Of course, larger fragments, e.g., 50-1500 nucleotides in length are also useful according to the present invention as are fragments corresponding to most, if not all, of the nucleotide sequence of the deposited cDNA or as shown in FIG. 1 (SEQ ID NO:1). By a fragment at least 20 nucleotides in length, for example, is intended fragments which include 20 or more contiguous bases from the nucleotide sequence of the deposited cDNA or the nucleotide sequence as shown in FIG. 1 (SEQ ID NO:1).

Preferred nucleic acid fragments of the present invention include nucleic acid molecules encoding: a polypeptide comprising the MOGp extracellular domain (predicted to constitute amino acid residues from about 30 to about 247 in FIG. 1 (SEQ ID NO:2)); a polypeptide comprising the MOGp transmembrane domain (predicted to constitute amino acid residues from about 248 to about 271 in FIG. 1 (SEQ ID NO:2)); a polypeptide comprising the MOGp intracellular domain (predicted to constitute amino acid residues from about 272 to about 331 in FIG. 1 (SEQ ID NO:2)); and a polypeptide comprising the MOGp extracellular and intracellular domains with all or part of the transmembrane domain deleted. As above with the leader sequence, the amino acid residues constituting the MOGp extracellular, transmembrane and intracellular domains have been predicted by computer analysis. Thus, as one of ordinary skill would appreciate, the amino acid residues constituting these domains may vary slightly (e.g., by about 1 to about 15 amino acids residues) depending on the criteria used to define each domain.

Preferred nucleic acid fragments of the present invention include nucleic acid molecules encoding epitope-bearing portions of the MOGp protein, the extracellular domain, the transmembrane domain, and the intracellular domain. In particular, such nucleic acid fragments of the present invention include nucleic acid molecules encoding: a polypeptide comprising amino acid residues from about 80 to about 113 in FIG. 1 (SEQ ID NO:2); a polypeptide comprising amino acid residues from about 282 to about 297 in FIG. 1 (SEQ ID NO:2); a polypeptide comprising amino acid residues from about 299 to about 331 in FIG. 1 (SEQ ID NO:2); a polypeptide comprising amino acid residues from about 46 to about 53 in FIG. 1 (SEQ ID NO:2); a polypeptide comprising amino acid residues from about 59 to about 65 in FIG. 1 (SEQ ID NO:2); a polypeptide comprising amino acid residues from about 71 to about 77 in FIG. 1 (SEQ ID NO:2); a polypeptide comprising amino acid residues from about 119 to about 125 in FIG. 1 (SEQ ID NO:2); a polypeptide comprising amino acid residues from about 130 to about 137 in FIG. 1 (SEQ ID NO:2); a polypeptide comprising amino acid residues from about 183 to about 190 in FIG. 1 (SEQ ID NO:2); a polypeptide comprising amino acid residues from about 211 to about 219 in FIG. 1 (SEQ ID NO:2); a polypeptide comprising amino acid residues from about 239 to about 248 in FIG. 1 (SEQ ID NO:2); and a polypeptide comprising amino acid residues from about 275 to about 280 in FIG. 1 (SEQ ID NO:2). The above polypeptide fragments are believed to be antigenic regions of the MOGp protein. Methods for determining other such epitope-bearing portions of the MOGp protein are described in detail below.

In addition, the present inventors have identified the following cDNA clones related to portions of SEQ ID NO. 1: HAFAV34R (SEQ ID NO. 11); HETBC89R (SEQ ID NO. 12); HRDDL76R (SEQ ID NO. 13); HRDDL35R (SEQ ID NO. 14); HRDDI47R (SEQ ID NO. 15); HRDDK16R (SEQ ID NO. 16); HRDDK03R (SEQ ID NO. 17); HRDDK54R (SEQ ID NO. 18); HRDBQ91R (SEQ ID NO. 19); HRDCB31R (SEQ ID NO. 20); HRDDL95R (SEQ ID NO. 21); HFCAE49F (SEQ ID NO. 22); and HTWAL13R (SEQ ID NO. 23).

The following public ESTs, which relate to portions of SEQ ID NO. 1 have also been identified: T91685 (SEQ ID NO. 24); AA303854 (SEQ ID NO. 25); T70127 (SEQ ID NO. 26); T86577 (SEQ ID NO. 27); AA337675 (SEQ ID NO. 28); T94934 (SEQ ID NO. 29); AA114263 (SEQ ID NO. 30); T92875 (SEQ ID NO. 31); AA484820 (SEQ ID NO. 32); T70246 (SEQ ID NO. 33); AA134341 (SEQ ID NO. 34); AA134342 (SEQ ID NO. 35); T94480 (SEQ ID NO. 36); T89056 (SEQ ID NO. 37); T86754 (SEQ ID NO. 38); and T98146 (SEQ ID NO. 39).

In another aspect, the invention provides an isolated nucleic acid molecule comprising a polynucleotide which hybridizes under stringent hybridization conditions to a portion of the polynucleotide in a nucleic acid molecule of the invention described above, for instance, the cDNA clone contained in ATCC Deposit No. 97709. By “stringent hybridization conditions” is intended overnight incubation at 42° C. in a solution comprising: 50% formamide, 5×SSC (150 mM NaCl, 15 mM trisodium citrate), 50 mM sodium phosphate (pH 7.6), 5× Denhardt's solution, 10% dextran sulfate, and 20 g/ml denatured, sheared salmon sperm DNA, followed by washing the filters in 0.1×SSC at about 65° C.

By a polynucleotide which hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30-70 nt of the reference polynucleotide. These are useful as diagnostic probes and primers as discussed above and in more detail below.

By a portion of a polynucleotide of “at least 20 nt in length,” for example, is intended 20 or more contiguous nucleotides from the nucleotide sequence of the reference polynucleotide (e.g., the deposited cDNA or the nucleotide sequence as shown in SEQ ID NO:1).

Of course, a polynucleotide which hybridizes only to a poly (A) sequence (such as the 3′ terminal poly(A) tract of the MOGp cDNA shown in FIG. 1 (SEQ ID NO:1)), or to a complementary stretch of T (or U) resides, would not be included in a polynucleotide of the invention used to hybridize to a portion of a nucleic acid of the invention, since such a polynucleotide would hybridize to any nucleic acid molecule containing a poly (A) stretch or the complement thereof (e.g., practically any double-stranded cDNA clone).

As indicated, nucleic acid molecules of the present invention which encode a MOGp polypeptide may include, but are not limited to those encoding the amino acid sequence of the mature polypeptide, by itself; the coding sequence for the mature polypeptide and additional sequences, such as those encoding the about 29 amino acid leader or secretory sequence, such as a pre-, or pro- or prepro-protein sequence; the coding sequence of the mature polypeptide, with or without the aforementioned additional coding sequences, together with additional, non-coding sequences, including for example, but not limited to introns and non-coding 5′ and 3′ sequences, such as the transcribed, non-translated sequences that play a role in transcription, mRNA processing, including splicing and polyadenylation signals, for example—ribosome binding and stability of mRNA; an additional coding sequence which codes for additional amino acids, such as those which provide additional functionalities. Thus, the sequence encoding the polypeptide may be fused to a marker sequence, such as a sequence encoding a peptide which facilitates purification of the fused polypeptide. In certain preferred embodiments of this aspect of the invention, the marker amino acid sequence is a hexa-histidine peptide, such as the tag provided in a pQE vector (Qiagen, Inc.), among others, many of which are commercially available. As described in Gentz et al., Proc. Natl. Acad. Sci. USA 86:821-824 (1989), for instance, hexa-histidine provides for convenient purification of the fusion protein. The “HA” tag is another peptide useful for purification which corresponds to an epitope derived from the influenza hemagglutinin protein, which has been described by Wilson et al., Cell 37: 767 (1984). As discussed below, other such fusion proteins include the MOGp fused to Fc at the N- or C-terminus.

The present invention further relates to variants of the nucleic acid molecules of the present invention, which encode portions, analogs or derivatives of the MOGp protein. Variants may occur naturally, such as a natural allelic variant. By an “allelic variant” is intended one of several alternate forms of a gene occupying a given locus on a chromosome of an organism. Genes II, Lewin, B., ed., John Wiley & Sons, New York (1985). Non-naturally occurring variants may be produced using art-known mutagenesis techniques.

Such variants include those produced by nucleotide substitutions, deletions or additions, which may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the MOGp protein or portions thereof. Also especially preferred in this regard are conservative substitutions.

Further embodiments of the invention include isolated nucleic acid molecules comprising a polynucleotide having a nucleotide sequence at least 90% identical, and more preferably at least 95%, 96%, 97%, 98% or 99% identical to (a) a nucleotide sequence encoding the full-length MOGp polypeptide having the complete amino acid sequence in FIG. 1 (SEQ ID NO:2), including the predicted leader sequence; (b) a nucleotide sequence encoding the full-length MOGp polypeptide without the N-terminal methionine having the amino acid sequence at positions 2-331 in FIG. 1 (SEQ ID NO:2); (c) a nucleotide sequence encoding the mature MOGp polypeptide (full-length polypeptide with the leader removed) having the amino acid sequence at positions 30-331 in FIG. 1 (SEQ ID NO:2); (c) a nucleotide sequence encoding the full-length MOGp polypeptide having the complete amino acid sequence including the leader encoded by the cDNA clone contained in ATCC Deposit No. 97709; (d) a nucleotide sequence encoding the mature MOGp polypeptide having the amino acid sequence encoded by the cDNA clone contained in ATCC Deposit No. 97709; (e) a nucleotide sequence encoding the MOGp extracellular domain; (f) a nucleotide sequence encoding the MOGp transmembrane domain; (g) a nucleotide sequence encoding the MOGp intracellular domain; and (h) a nucleotide sequence complementary to any of the nucleotide sequences in (a), (b), (c), (d), (e), (f) or (g).

By a polynucleotide having a nucleotide sequence at least, for example, 95% “identical” to a reference nucleotide sequence encoding a MOGp polypeptide is intended that the nucleotide sequence of the polynucleotide is identical to the reference sequence except that the polynucleotide sequence may include up to five point mutations per each 100 nucleotides of the reference nucleotide sequence encoding the MOGp polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 90%, 95%, 96%, 97%, 98% or 99% identical to, for instance, the nucleotide sequence shown in FIG. 1 or to the nucleotides sequence of the deposited cDNA clone can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

The present application is directed to nucleic acid molecules at least 90%, 95%, 96%, 97%, 98% or 99% identical to the nucleic acid sequence shown in FIG. 1 (SEQ ID NO:1) or to the nucleic acid sequence of the deposited cDNA, irrespective of whether they encode a polypeptide having MOGp activity. This is because even where a particular nucleic acid molecule does not encode a polypeptide having MOGp activity, one of skill in the art would still know how to use the nucleic acid molecule, for instance, as a hybridization probe or a polymerase chain reaction (PCR) primer. Uses of the nucleic acid molecules of the present invention that do not encode a polypeptide having MOGp activity include, inter alia, (1) isolating the MOGp gene or allelic variants thereof in a cDNA library; (2) in situ hybridization (e.g., “FISH”) to metaphase chromosomal spreads to provide precise chromosomal location of the MOGp gene, as described in Verma et al., Human Chromosomes: A Manual of Basic Techniques, Pergamon Press, New York (1988); and Northern Blot analysis for detecting MOGp mRNA expression in specific tissues.

Preferred, however, are nucleic acid molecules having sequences at least 90%, 95%, 96%, 97%, 98% or 99% identical to the nucleic acid sequence shown in FIG. 1 (SEQ ID NO:1) or to the nucleic acid sequence of the deposited cDNA which do, in fact, encode a polypeptide having MOGp protein activity.

By “a polypeptide having MOGp activity” is intended polypeptides exhibiting activity similar, but not necessarily identical, to an activity of the MOGp protein of the invention (either the full-length protein, the mature protein, or soluble derivatives thereof), as measured in at least one biological assay. For example, MOGp protein activity can be determined using an immunological assay that measures binding to MOG or MOGp antibodies. Antibodies that specifically bind to MOGp can be incubated with a sample to be tested for MOGp biological activity and binding of the antibodies to an antigen in that sample is indicative of the presence of MOGp activity. Methods of assaying for the presence of the antigen having a specified reactivity are well known in the art. For example, Western blots, enzyme-linked immunosorbent assays (ELISA), radioimmunoassays and the like can be used. Methods of obtaining specific antibodies to MOGp are also well known in the art based on the information provided herein. This immunological reactivity is useful for diagnosis of tumors, inflammation or MS.

Alternatively, a protein having MOGp activity, such as soluble fragments of MOGp, can be used to disrupt or compete for the binding of MOGp, to its ligand or to a specific antibody. For example, a polypeptide having MOGp activity may affect the interaction between MOGp and other proteins. This interaction can be associated with disease states such as cancer, inflammation, or MS. Thus, a MOGp polypeptide has MOGp activity if it ameliorates these disease states by antagonizing binding of MOGp to its ligand or an antibody.

Thus, “a polypeptide having MOGp protein activity” includes polypeptides that exhibit MOGp activity, in at least one of the above-described assays.

Of course, due to the degeneracy of the genetic code, one of ordinary skill in the art will recognize that a large number of the nucleic acid molecules having a sequence at least 90%, 95%, 96%, 97%, 98%, or 99% identical to the nucleic acid sequence of the deposited cDNA or the nucleic acid sequence shown in FIG. 1 (SEQ ID NO:1) will encode a polypeptide “having MOGp protein activity.” In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having MOGp protein activity. This is because the skilled artisan is fully aware of amino acid substitutions that are either less likely or not likely to significantly effect protein function (e.g., replacing one aliphatic amino acid with a second aliphatic amino acid, as defined infra).

For example, guidance concerning how to make phenotypically silent amino acid substitutions is provided in Bowie, J. U. et al., “Deciphering the Message in Protein Sequences: Tolerance to Amino Acid Substitutions,” Science 247:1306-1310 (1990), wherein the authors indicate that proteins are surprisingly tolerant of amino acid substitutions.

Vectors and Host Cells

The present invention also relates to vectors which include the isolated DNA molecules of the present invention, host cells which are genetically engineered with the recombinant vectors, and the production of MOGp polypeptides or fragments thereof by recombinant techniques.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The DNA insert should be operatively linked to an appropriate promoter, such as the phage lambda PL promoter, the E. coli lac, trp and tac promoters, the SV40 early and late promoters and promoters of retroviral LTRs, to name a few. Other suitable promoters will be known to the skilled artisan. The expression constructs will further contain sites for transcription initiation, termination and, in the transcribed region, a ribosome binding site for translation. The coding portion of the mature transcripts expressed by the constructs will preferably include a translation initiating at the beginning and a termination codon (UAA, UGA or UAG) appropriately positioned at the end of the polypeptide to be translated.

As indicated, the expression vectors will preferably include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture mediums and conditions for the above-described host cells are known in the art.

Among vectors preferred for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

In a more specific embodiment, the nucleic acid molecules of the present invention, e.g., isolated nucleic acids comprising a polynucleotide having a nucleotide sequence encoding a MOGp polypeptide or fragments thereof, are not the sequence of nucleotides, the nucleic acid molecules (e.g., clones), or the nucleic acid inserts identified in one or more of the following GenBank Accession Reports: T91685, AA303854, T70127, T86577, AA337675, T94934, AA114263, T92875, AA484820, T70246, AA134342, AA134341, T94480, T89056, T86754, and T98146, all of which are incorporated herein by reference.

In other embodiments this invention provides an isolated nucleic acid molecule comprising a MOGp structural gene operably linked to a heterologous promoter. As used herein, the term “a MOGp structural gene” refers to a nucleotide sequence at least 95% identical to one of the following nucleotide sequences:

(a) a nucleotide sequence encoding the MOGp polypeptide having the complete amino acid sequence in FIG. 1 (SEQ ID NO:2);

(b) a nucleotide sequence encoding the MOGp polypeptide having the amino acid sequence at positions 2-331 in FIG. 1 (SEQ ID NO:2);

(c) a nucleotide sequence encoding the mature MOGp polypeptide having the amino acid sequence at positions 30-331 in FIG. 1 (SEQ ID NO:2);

(d) a nucleotide sequence encoding the MOGp polypeptide having the complete amino acid sequence encoded by the cDNA clone contained in ATCC Deposit No. 97709;

(e) a nucleotide sequence encoding the mature MOGp polypeptide having the amino acid sequence encoded by the cDNA clone contained in ATCC Deposit No. 97709; or

(f) a nucleotide sequence complementary to any of the nucleotide sequences in (a), (b), (c), (d), or (e).

In more preferred embodiments, the MOGp structural gene is 96%, 97%, 98%, 99%, or 100% identical to one or more of nucleotide sequences (a)-(f), supra.

As used herein, the term “operably linked,” when used in the context of a linkage between a structural gene and an expression control sequence, e.g., a promoter, refers to the position and orientation of the expression control sequence relative to the structural gene so as to permit expression of the structural gene in any host cell. For example, an operable linkage would maintain proper reading frame and would not introduce any in frame stop codons.

As used herein, the term “heterologous promoter,” refers to a promoter not normally and naturally associated with the structural gene to be expressed. For example, in the context of expression of a MOGp polypeptide, a heterologous promoter would be any promoter other than an endogenous promoter associated with the MOGp gene in non-recombinant human chromosomes. In specific embodiments of this invention, the heterologous promoter is not a prokaryotic or bacteriophage promoter, such as the lac promoter, T3 promoter, or T7 promoter. In other embodiments, the heterologous promoter is a eukaryotic promoter.

This invention also provides an isolated nucleic acid molecule comprising a MOGp structural gene operably linked to a heterologous promoter, wherein said isolated nucleic acid molecule does not encode a fusion protein comprising the MOGp structural gene or a fragment thereof. In particular embodiments the isolated nucleic acid molecule does not encode a beta-galactosidase—MOGp fusion protein.

This invention further provides an isolated nucleic acid molecule comprising a MOGp structural gene operably linked to a heterologous promoter, wherein said isolated nucleic acid molecule is capable of expressing a MOGp polypeptide when used to transform an appropriate host cell. In particular embodiments, the MOGp polypeptide does not contain and is not covalently linked to an amino acid sequence encoded by the 5′ untranslated portion of the MOGp gene, e.g., nucleotides 1-47 of FIG. 1 (SEQ ID NO. 1), or a fragment thereof.

This invention also provides an isolated nucleic acid molecule comprising a polynucleotide having a nucleotide sequence at least 95% identical to a sequence encoding a MOGp polypeptide having the amino acid sequence of SEQ ID NO. 2, wherein said isolated nucleic acid molecule does not contain a nucleotide sequence at least 90% identical to the 3′ untranslated region of FIG. 1 (nucleotides 1044-1512), or a fragment of the 3′ untranslated region greater than 25, 50, 75, 100, 125, 150, 175, 200, 225, 250, 275, 300, 325, 350, 375, 400, 425, or 450 bp in length. In other embodiments, said isolated nucleic acid molecule does not contain a nucleotide sequence at least 91%, 92%, 93%, 94%, 95%, 96%, 97%, 98%, 99%, or 100% identical to the 3′ untranslated region of FIG. 1 (nucleotides 1044-1512).

This invention further provide an isolated nucleic acid molecule comprising a polynucleotide having a nucleotide sequence at least 95% identical to a sequence encoding a MOGp polypeptide having the amino acid sequence of SEQ ID NO. 2, wherein said isolated nucleic acid molecule does not contain a nucleotide sequence at least 90% identical to the 5′ untranslated region of FIG. 1 (nucleotides 1-47), or a fragment of the 5′ untranslated region greater than 10, 20, 30, or 40 kb. In other embodiments, said isolated nucleic acid molecule does not contain a nucleotide sequence at least 91%, 92%, 93%, 94%, 95%, 96%, 97%, 98%, 99%, or 100% identical to the 5′ untranslated region of FIG. 1 (nucleotides 1-47).

In addition to the use of expression vectors in the practice of the present invention, the present invention further includes novel expression vectors comprising operator and promoter elements operatively linked to nucleotide sequences encoding a protein of interest. One example of such a vector is pHE4-5 which is described in detail below.

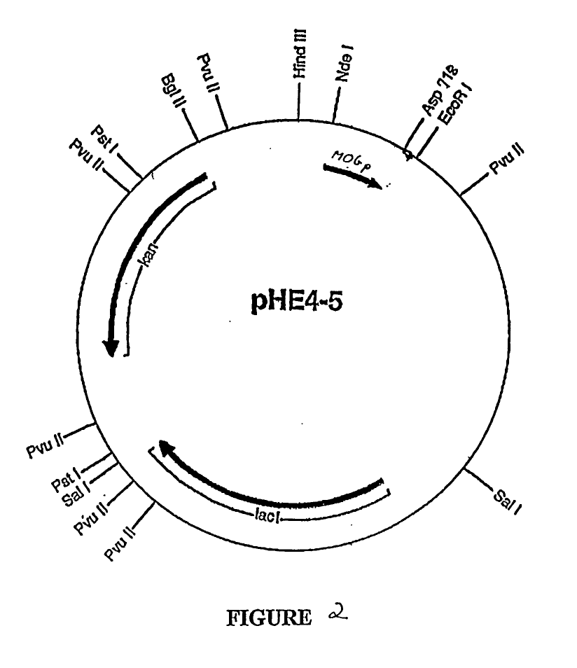

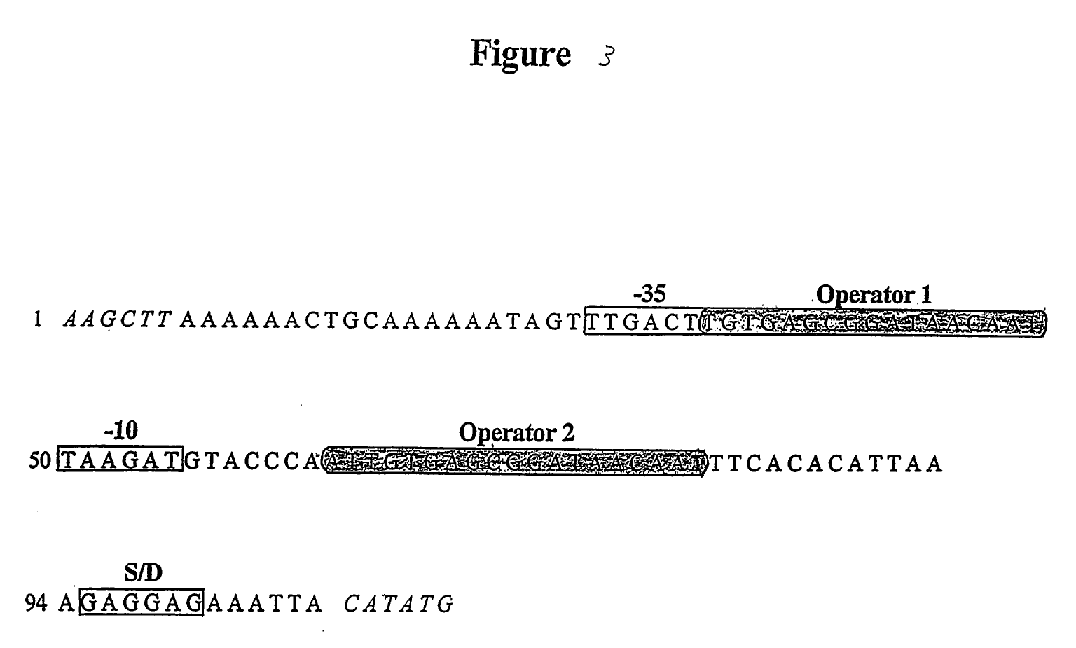

As summarized in FIGS. 2 and 3, components of the pHE4-5 vector (SEQ ID NO:9) include: 1) a neomycinphosphotransferase gene as a selection marker, 2) an E. coli origin of replication, 3) a T5 phage promoter sequence, 4) two lac operator sequences, 5) a Shine-Delgarno sequence, 6) the lactose operon repressor gene (lacIq). The origin of replication (oriC) is derived from pUC19 (LTI, Gaithersburg, Md.). The promoter sequence and operator sequences were made synthetically. Synthetic production of nucleic acid sequences is well known in the art. CLONTECH 95/96 Catalog, pages 215-216, CLONTECH, 1020 East Meadow Circle, Palo Alto, Calif. 94303. A nucleotide sequence encoding MOGp (SEQ ID NO:1), is operatively linked to the promoter and operator by inserting the nucleotide sequence between the NdeI and Asp718 sites of the pHE4-5 vector.

As noted above, the pHE4-5 vector contains a lacIq gene. LacIq is an allele of the lacI gene which confers tight regulation of the lac operator. Amann, E. et al., Gene 69:301-315 (1988); Stark, M., Gene 51:255-267 (1987). The lacIq gene encodes a repressor protein which binds to lac operator sequences and blocks transcription of down-stream (i.e., 3′) sequences. However, the lacIq gene product dissociates from the lac operator in the presence of either lactose or certain lactose analogs, e.g., isopropyl B-D-thiogalactopyranoside (IPTG). MOGp thus is not produced in appreciable quantities in uninduced host cells containing the pHE4-5 vector. Induction of these host cells by the addition of an agent such as IPTG, however, results in the expression of the MOGp coding sequence.

The promoter/operator sequences of the pHE4-5 vector (SEQ ID NO:10) comprise a T5 phage promoter and two lac operator sequences. One operator is located 5′ to the transcriptional start site and the other is located 3′ to the same site. These operators, when present in combination with the lacIq gene product, confer tight repression of down-stream sequences in the absence of a lac operon inducer, e.g., IPTG. Expression of operatively linked sequences located down-stream from the lac operators may be induced by the addition of a lac operon inducer, such as IPTG. Binding of a lac inducer to the lacIq proteins results in their release from the lac operator sequences and the initiation of transcription of operatively linked sequences. Lac operon regulation of gene expression is reviewed in Devlin, T., TEXTBOOK OF BIOCHEMISTRY WITH CLINICAL CORRELATIONS, 4th Edition (1997), pages 802-807.

The pHE4 series of vectors contain all of the components of the pHE4-5 vector except for the MOGp coding sequence. Features of the pHE4 vectors include optimized synthetic T5 phage promoter, lac operator, and Shine-Delagarno sequences. Further, these sequences are also optimally spaced so that expression of an inserted gene may be tightly regulated and high level of expression occurs upon induction.

Among known bacterial promoters suitable for use in the production of proteins of the present invention include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous Sarcoma Virus (RSV), and metallothionein promoters, such as the mouse metallothionein-I promoter.

The pHE4-5 vector also contains a Shine-Delgarno sequence 5′ to the AUG initiation codon. Shine-Delgarno sequences are short sequences generally located about 10 nucleotides up-stream (i.e., 5′) from the AUG initiation codon. These sequences essentially direct prokaryotic ribosomes to the AUG initiation codon.

Thus, the present invention is also directed to expression vector useful for the production of the proteins of the present invention. This aspect of the invention is exemplified by the pHE4-5 vector (SEQ ID NO:9).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986).

The polypeptide may be expressed in a modified form, such as a fusion protein, and may include not only secretion signals, but also additional heterologous functional regions. In addition, the DNA can be appropriately modified, e.g., by insertion of in-frame stop codons, to express only the soluble extracellular domain. If desired, the intracellular domain or the transmembrane domain can also be differentially expressed. In addition, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art.

A preferred fusion protein comprises a heterologous region from immunoglobulin that is useful to solubilize proteins. For example, EP-A-O 464 533 (Canadian counterpart 2045869) discloses fusion proteins comprising various portions of constant region of immunoglobin molecules together with another human protein or part thereof. In many cases, the Fe part in a fusion protein is thoroughly advantageous for use in therapy and diagnosis and thus results, for example, in improved pharmacokinetic properties (EP-A 0232 262). On the other hand, for some uses it would be desirable to be able to delete the Fe part after the fusion protein has been expressed, detected and purified in the advantageous manner described. This is the case when Fe portion proves to be a hindrance to use in therapy and diagnosis, for example when the fusion protein is to be used as antigen for immunizations. In drug discovery, for example, human proteins, such as, hIL5- has been fused with Fe portions for the purpose of high-throughput screening assays to identify antagonists of hIL-5. See, D. Bennett et al., J. Molec. Recog., 8:52-58 (1995) and K. Johanson et al., J. Biol. Chem., 270:(16):9459-9471 (1995).

The MOGp protein can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. When full-length MOGp or fragments of MOGp comprising the transmembrane domain, are expressed, the protein is usually solubilized in a buffer containing an effective concentration of a detergent. Examples of suitable detergents include Triton, Tween, and deoxycholate. Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

MOGp Polypeptides and Fragments

The invention further provides an isolated MOGp polypeptide having the amino acid sequence encoded by the deposited cDNA, or the amino acid sequence in FIG. 1 (SEQ ID NO:2), or a peptide or polypeptide comprising a portion of the above polypeptides.

More particularly this invention provides membrane forms of the MOGp polypeptide that lacks all or a portion of the leader sequence. For example, MOGp polypeptides having amino acids 16-331, 17-331, 18-331, 19-331, 20-331, 21-331, 22-331, 23-331, 24-331, 25-331, 26-331, 27-331, 28-331, 29-331, 30-331, 31-331, 32-331, 33-331, 34-331, 35-331, 36-331, 37-331, 38-331, 39-331, 40-331, 41-331, 42-331, 43-331, 44-331, 45-331, 46-331, 47-331, 48-331, 49-331, and 50-331 are contemplated. In addition, soluble forms of the MOGp polypeptide lacking all or a part of the leader sequence are contemplated, such as polypeptides having amino acids 16-247, 17-247, 18-247, 19-247, 20-247, 21-247, 22-247, 23-247, 24-247, 25-247, 26-247, 27-247, 28-247, 29-247, 30-247, 31-247, 32-247, 33-247, 34-247, 35-247, 36-247, 37-247, 38-247, 39-247, 40-247, 41-247, 42-247, 43-247, 44-247, 45-247, 46-247, 47-247, 48-247, 49-247, and 50-247. Also contemplated are nucleic acid molecules encoding these membrane-bound and soluble MOGp polypeptides and vectors and host cells comprising them.

While these polypeptides can be routinely tested for biological activity using the teachings found herein, it is believed these forms of MOGp should retain biological activity. Disulfide bonds formed between cysteine residues are often involved in maintaining secondary structure and activity. The first cysteine residue that is conserved between MOG and MOGp is retained in each of these forms. Consequently, biological activity should also be retained.

This invention also provides forms of the MOGp polypeptides lacking the N-terminal methionine, nucleic acids encoding them, and vectors and host cells comprising them.

It will be recognized in the art that some amino acid sequences of the MOGp polypeptide can be varied without significant effect of the structure or function of the protein. If such differences in sequence are contemplated, it should be remembered that there will be critical areas on the protein which determine activity.

Thus, the invention further includes variations of the MOGp polypeptide which show substantial MOGp polypeptide activity or which include regions of MOGp protein such as the protein portions discussed below. Such mutants include deletions, insertions, inversions, repeats, and type substitutions, as indicated above, can be found in Bowie et al., Science 247:1306-1310 (1990).

Thus, the fragment, derivative or analog of the polypeptide of FIG. 1 (SEQ ID NO:2), or that encoded by the deposited cDNA, may be (i) one in which one or more amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one which the mature polypeptide is fused with another compound, such as a compound to increase the half-life of the polypeptide (for example, polyethylene glycol), or (iv) one in which the additional amino acids are fused to the mature polypeptide, such as an IgG Fc fusion region peptide or leader or secretory sequence or a sequence which is employed for purification of the mature polypeptide or a proprotein sequence. Such fragments, derivatives and analogs are deemed to be within the scope of those skilled in the art from the teachings herein.

Of particular interest are substitutions of charged amino acids with another charged amino acid and with neutral or negatively charged amino acids. The latter results in proteins with reduced positive charge to improve the characteristics of the MOGp protein. The prevention of aggregation is highly desirable. Aggregation of proteins not only results in a loss of activity but can also be problematic when preparing pharmaceutical formulations, because they can be immunogenic. (Pinckard et al., Clin Exp. Immunol. 2:331-340 (1967); Robbins et al., Diabetes 36:838-845 (1987); Cleland et al., Crit. Rev. Therapeutic Drug Carrier Systems 10:307-377 (1993)).

The replacement of amino acids can also change the selectively of binding to cell surface receptors. Ostade et al., Nature 361:266-268 (1993) describes certain mutations resulting in selective binding of TNF-α to only one of the two known types of TNF receptors. Thus, the MOGp protein of the present invention may include one or more amino acid substitutions, deletions or additions, either from natural mutations or human manipulation.

As indicated, changes are preferably of a minor nature, such as conservative amino acid substitutions that do not significantly affect the folding or activity of the protein (see Table 1).

TABLE 1
Conservative Amino Acid Substitutions.

| | |
|---|---|
| AROMATIC | Phenylalanine |
| | Tryptophan |
| | Tyrosine |
| HYDROPHOBIC | Leucine |
| | Isoleucine |
| | Valine |
| POLAR | Glutamine |
| | Asparagine |
| BASIC | Arginine |
| | Lysine |
| | Histidine |
| ACIDIC | Aspartic Acid |
| | Glutamic Acid |
| SMALL | Alanine |
| | Serine |
| | Threonine |
| | Methionine |
| | Glycine |

Of course, the number of amino acid substitutions a skilled artisan would make depends on many factors, including those described above. Generally speaking, the number of amino acid substitutions for any given MOGp polypeptide will not be more than 50, 40, 30, 20, 10, 5, or 3.

Amino acids in the MOGp protein of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro, or in vitro proliferative activity. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al., Science 255:306-312 (1992)).

The polypeptides of the present invention are preferably provided in an isolated form. By “isolated polypeptide” is intended a polypeptide removed from its native environment. Thus, a polypeptide produced and/or contained within a recombinant host cell is considered isolated for purposes of the present invention. Also intended as an “isolated polypeptide” are polypeptides that have been purified, partially or substantially, from a recombinant host cell or from a native source. For example, a recombinantly produced version of the MOGp polypeptide can be substantially purified by the one-step method described in Smith and Johnson, Gene 67:31-40 (1988).

The polypeptides of the present invention include the polypeptide encoded by the deposited cDNA including the leader, the mature polypeptide encoded by the deposited the cDNA minus the leader (i.e., the mature protein), the polypeptide of FIG. 1 (SEQ ID NO:2) including the leader, the polypeptide of FIG. 1 (SEQ ID NO:2) minus the leader, the extracellular domain, the transmembrane domain, and the intracellular domain, as well as polypeptides which have at least 90% similarity, more preferably at least 95% similarity, and still more preferably at least 96%, 97%, 98% or 99% similarity to those described above. Further polypeptides of the present invention include polypeptides at least 80% identical, more preferably at least 90% or 95% identical, still more preferably at least 96%, 97%, 98% or 99% identical to the polypeptide encoded by the deposited cDNA, to the polypeptide of FIG. 1 (SEQ ID NO:2), and also include portions of such polypeptides with at least 30 amino acids and more preferably at least 50 amino acids.

By a polypeptide having an amino acid sequence at least, for example, 95% “identical” to a reference amino acid sequence of a MOGp polypeptide is intended that the amino acid sequence of the polypeptide is identical to the reference sequence except that the polypeptide sequence may include up to five amino acid alterations per each 100 amino acids of the reference amino acid of the MOGp receptor. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequence shown in FIG. 1 (SEQ ID NO:2) or to the amino acid sequence encoded by deposited cDNA clone can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set such that the percentage of identity is calculated over the full length of the reference amino acid sequence and that gaps in homology of up to 5% of the total number of amino acid residues in the reference sequence are allowed.

The polypeptide of the present invention could be used as a molecular weight marker on SDS-PAGE gels or on molecular sieve gel filtration columns using methods well known to those of skill in the art.

In another aspect, the invention provides a peptide or polypeptide comprising an epitope-bearing portion of a polypeptide of the invention. The epitope of this polypeptide portion is an immunogenic or antigenic epitope of a polypeptide described herein. An “immunogenic epitope” is defined as a part of a protein that elicits an antibody response when the whole protein is the immunogen. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad. Sci. USA 81:3998-4002 (1983).

Preferred epitopes of MOGp include portions of the N-terminal domain. For example, the encephalitogenic T cell response to the N-terminal domain of MOG was found to recognize two distinct epitopes: MOG₁₋₂₀ and MOG₃₅₋₅₅, Adelmann et al., J. Neuroimmunol 63:17-87 (1995). The core of this epitope is between residues 9-15. Amor et al., J. Immunol. 156:3000-3008 (1996). MOGp shares significant sequence homology with MOG within these regions. In specific embodiments of this invention, a polypeptides comprising amino acids 1-125 are provided. In other embodiments of this invention, polypeptides comprising amino acids 80-113; 282-297; 299-331; 46-53; 59-65; 71-77; 119-125; 130-137; 183-190; 211-219; 239-248; and 275-280 of MOGp are provided.

As to the selection of peptides or polypeptides bearing an antigenic epitope, i.e., that contain a region of a protein molecule to which an antibody can bind, it is well known in that art that relatively short synthetic peptides that mimic part of a protein sequence are routinely capable of eliciting an antiserum that reacts with the partially mimicked protein. See, for instance, Sutcliffe, J. G., Shinnick, T. M., Green, N. and Learner, R. A., Antibodies that react with predetermined sites on proteins, Science 219:660-666 (1983). Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals.

Antigenic epitope-bearing peptides and polypeptides of the invention are therefore useful to raise antibodies, including monoclonal antibodies, that bind specifically to a polypeptide of the invention. See, for instance, Wilson et al., Cell 37:767-778 (1984) at 777.

Antigenic epitope-bearing peptides and polypeptides of the invention preferably contain a sequence of at least seven, more preferably at least nine and most preferably between at least about 15 to about 30 amino acids contained within the amino acid sequence of a polypeptide of the invention.

Non-limiting examples of antigenic polypeptides or peptides that can be used to generate MOGp-specific antibodies include: a polypeptide obtained from the N-terminus of MOGp. In FIG. 1 (SEQ ID. NO:2). In more specific embodiments of this invention the polypeptide comprises amino acid residues 38 to 43 and residues 35-55 in FIG. 1 (SEQ ID. NO:2). Other non-limiting examples include a polypeptide comprising amino acid residues from about 80-113; 282-297; 299-331; 46-53; 59-65; 71-77; 119-125; 130-137; 183-190; 211-219; 239-248; and 275-280 in FIG. 1 (SEQ ID NO:2). As indicated above, the inventors have determined that the above polypeptide fragments are antigenic regions of the MOGp protein.

The epitope-bearing peptides and polypeptides of the invention may be produced by any conventional means. Houghten, R. A., General Method for the Rapid Solid-phase Synthesis of Large Numbers of Peptides: Specificity of Antigen-antibody Interaction at the Level of Individual Amino Acids. Proc. Natl. Acad. Sci. USA 82:5131-5135 (1985). This “Simultaneous Multiple Peptide Synthesis (SMPS)” process is further described in U.S. Pat. No. 4,631,211 to Houghten et al. (1986).

As one of skill in the art will appreciate, MOGp polypeptides of the present invention and the epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EPA 394,827; Traunecker et al., Nature 331:84-86 (1988)). Fusion proteins that have a disulfide-linked dimeric structure due to the IgG part can also be more efficient in binding and neutralizing other molecules than the monomeric MOGp protein or protein fragment alone (Fountoulakis et al., J. Biochem. 270:3958-3964 (1995)).

In addition, it is known in the art for many proteins, including the mature form(s) of a secreted protein, that one or more amino acids may be deleted from the N-terminus without substantial loss of biological function. However, even if deletion of one or more amino acids from the N-terminus results in a modification or loss of one or more biological functions, other biological activities may still be retained. For example, the ability of the shortened protein to induce or bind to antibodies which recognize the complete or mature protein will generally be retained when less than the majority of residues are removed from the N-terminus. Whether these immunological activities are retained can readily be determined by routine methods described herein or otherwise known in the art. Accordingly, the present invention further provides polypeptides having one or more residues deleted from the N-terminus of the amino acid sequence of FIG. 1, and polynucleotides encoding such polypeptides.

Cancer Diagnosis and Prognosis

It is believed that certain tissues in mammals with cancer express significantly enhanced levels of the MOGp protein and mRNA encoding the MOGp when compared to a corresponding “standard” mammal, i.e., a mammal of the same species not having the cancer. Further, it is believed that enhanced levels of the MOGp protein can be detected in certain body fluids (e.g., sera, plasma, urine and spinal fluid) from mammals with cancer when compared to sera from mammals of the same species not having cancer. Thus, the invention provides a diagnostic method useful for tumor diagnosis, which involves assaying the expression level of the gene encoding the MOGp protein in mammalian cells or body fluid and comparing the gene expression level with a standard MOGp protein gene expression level, whereby an increase in the gene expression level over the standard is indicative of certain tumors.

Where a tumor diagnosis has already been made according to conventional methods, the present invention is useful as a prognostic indicator, whereby patients exhibiting enhanced MOGp gene expression will experience a worse clinical outcome relative to patients expressing the gene at a lower level.

By “assaying the expression level of the gene encoding the MOGp protein” is intended qualitatively or quantitatively measuring or estimating the level of the MOGp protein or the level of the mRNA encoding the MOGp protein in a first biological sample either directly (e.g., by determining or estimating absolute protein level or mRNA level) or relatively (e.g., by comparing to the MOGp protein level or mRNA level in a second biological sample).

Preferably, the MOGp protein level or mRNA level in the first biological sample is measured or estimated and compared to a standard MOGp protein level or mRNA level, the standard being taken from a second biological sample obtained from an individual not having the cancer. As will be appreciated in the art, once a standard MOGp protein level or mRNA level is known, it can be used repeatedly as a standard for comparison.

By “biological sample” is intended any biological sample obtained from an individual, cell line, tissue culture, or other source which may contain MOGp protein or mRNA. Biological samples include peripheral blood lymphocytes and related tissue, such as bone marrow.

The present invention is useful for detecting cancer in mammals. In particularly the invention is useful during diagnosis of the following types of cancers in mammals: breast, ovarian, prostate, bone, liver, lung, pancreatic, and spleenic. Preferred mammals include monkeys, apes, cats, dogs, cows, pigs, horses, rabbits and humans. Particularly preferred are humans.

Total cellular RNA can be isolated from a biological sample using the single-step guanidinium-thocyanate-phenol-chloroform method described in Chomczynski and Sacchi, Anal. Biochem. 162:156-159 (1987). Levels of mRNA encoding the MOGp protein are then assayed using any appropriate method. These include Northern blot analysis (Harada et al., Cell 63:303-312 (1990)), S1 nuclease mapping (Fujita et al., Cell 49:357-367 (1987)), the polymerases chain reaction (PCR), reverse transcription in combination with the polymerases chain reaction (RT-PCR) (Fujita et al., Cell 49:357-367 (1987)), and reverse transcription in combination with the ligase chain reaction (RT-LCR).

Assaying MOGp protein levels in a biological sample can occur using antibody-based techniques. For example, MOGp protein expression in tissues can be studied with classical immunohistological methods. (Jalkanen, M., et al., J. Cell. Biol. 101:976-985 (1985); Jalkanen, M., et al., J. Cell. Biol. 105:3087-3096 (1987)). Other antibody-based methods useful for detecting MOGp gene expression include immunoassays, such as the enzyme linked immunosorbent assay (ELISA) and the radioimmunoassays (RIA).

Suitable labels are known in the art and include enzyme labels, such as glucose oxidase, and radioisotopes, such as iodine (¹²⁵I, ¹²¹I), carbon (¹⁴C), sulpher (³⁵S), tritium (³H), indium (¹¹²In), and technetium (^{99m}Tc), and fluorescent lavels, such as fluorescein and rhodamine, and biotin.

Multiple Sclerosis (MS) Diagnosis and Prognosis

It is believed that the presence of antibody to MOGp at enhanced levels is associated with the development of MS, when compared to the level of antibody in a corresponding “standard” subject, i.e., a subject not having MS. Further, it is believed that enhanced levels of anti-MOGp can be detected in certain body fluids, e.g., sera, plasma, urine, and spinal fluid from mammals with MS when compared to these body fluids in subjects not having MS. Thus, the invention provides a diagnostic method for MS, which involves assaying for the presence of anti-MOGp antibody in mammalian cells or body fluid and comparing the level of antibody obtained with a standard, where an increase in the concentration of antibody over standard is indicative of MS.

Where a diagnosis of MS has already been made according to conventional methods, the present invention is useful as a prognostic indicator, whereby patients exhibiting enhanced presence of antibody to MOGp will experience a worse clinical outcome relative to patients expressing the gene at a lower level.

By “assaying the amount of anti-MOG antibody” is intended to refer to qualitatively or quantitatively measuring or estimating the level of anti-MOGp antibody in a first biological sample either directly (e.g., by determining or estimating absolute anti-MOGp antibody level) or relatively (e.g., by comparing the anti-MOGp level in the sample to the anti-MOGp antibody level in a second biological sample).

Preferably, the anti-MOGp antibody level in the first biological sample is measured or estimated and compared to a standard anti-MOGp antibody level, the standard being taken from a second biological sample obtained from an individual not having MS. As will be appreciated in the art, once a standard anti-MOGp antibody level is known, it can be used repeatedly as a standard for comparison.

By “biological sample,” in the context of MS diagnosis or prognosis, is intended any biological sample obtained from an individual, cell line, tissue culture, or other source which may contain anti-MOGp antibody protein or mRNA. Biological samples include mammalian body fluids (such as sera, plasma, urine, synovial fluid and spinal fluid) which may contain antibody to MOGp protein, and nerve tissue. Methods for obtaining tissue biopsies and body fluids from mammals are well known in the art.

Assaying anti-MOGp antibody levels in a biological sample can be performed using any of a variety of art-known methods. For example, immunoassays, such as the enzyme linked immunosorbent assay (ELISA) and radioimmunoassays (RIA), appropriately modified to detect antibody instead of antigen, can be used.

An indirect ELISA to detect anti-MOGp antibodies can be carried out by coating the wells of microtiter plates with antigen, incubating the coated plates with the sample to be assayed, and washing away the unbound antibodies. A solution containing a developing reagent, e.g., alkaline phosphatase conjugated to protein A, protein G, or antibodies against the test solution antibodies, is then added to the plate. After incubation, unbound conjugate is washed away and substrate solution is added. After a second incubation, the amount of substrate hydrolyzed is assessed with a spectrophotometer or spectrofluorometer. The measured amount is proportional to the amount of specific antibody in the test solution. For a more detailed discussion of this assay see Current Protocols in Molecular Biology, F. M. Ausubel et al. eds., at 11.2.2 (1991).

A specific antibody titer of a sample to be tested can be determined using a solid-phase radioimmunoassay. The sample is serially diluted and incubated in microtiter wells previously coated with MOGp. Unbound antibody is washed away. Bound antibody is detected by employing labeled, e.g., with ¹²⁵I, anti-immunoglobulin antibodies. The amount of specific antibody in the sample is then determined from a standard curve generated from a specific antibody of known concentration. Such an antibody can be obtained as described infra. For a more detailed discussion of this assay see Current Protocols in Molecular Biology, F. M. Ausubel et al. eds., at 11.16.1(1993).

The present invention is useful for detecting MS in mammals. Preferred mammals include monkeys, apes, cats, dogs, cows, pigs, horses, rabbits and humans. Particularly preferred are humans.

Method of Detecting Oligodendrocyte Maturation and Diagnosing Demyelinating Disease

The presence of MOGp on the surface of oligodendrocytes is believed to be associated with oligodendrocyte maturation. Therefore, MOGp can be used as a surface marker for oligodendrocyte maturation. Samples containing mature oligodendrocytes can be identified by significantly enhanced levels of MOGp protein or mRNA encoding MOGp protein when compared to a corresponding “standard” not containing mature oligodendrocytes. Since oligodendrocytes are responsible for the synthesis and maintenance of myelin in the CNS, the absence of mature oligodendrocytes in myelin tissue samples would correlate with demyelinating disease. Moreover, it is believed that MOGp itself is associated with the production of myelin. Therefore, the absence of MOGp per se should also correlate with demyelinating disease.

Thus, the invention also provides a diagnostic method useful for diagnosing demyelinating disease, which involves assaying the expression level of the gene encoding MOGp in mammalian cells such as oligodendrocytes and comparing the gene expression level with a standard MOGp gene expression levels, whereby a decrease in MOGp expression is indicative of demyelinating disease.

The present invention is useful for detecting demyelinating disease in mammals. Preferred mammals include monkeys, apes, cats, dogs, cows, pigs, horses, rabbits, and humans.

Therapeutics

It is believed that MOGp is associated with the development of an immune response. Therefore, any molecule capable of antagonizing the binding of MOGp to its ligand may be useful for treating inflammation. For example, anti-MOGp antibodies, soluble derivatives of MOGp, and solubilized MOGp can be used. Suitable soluble derivatives of MOGp include soluble fragments comprising the entire extracellular domain and portions thereof capable of treating inflammation.

An autoimmune response to myelin is associated with MS, resulting in the degradation of the myelin sheath. An important target for the autoantibody is MOG, which is exposed as the outermost lamellae of the myelin sheath and the oligodendrocyte plasma membrane. Therefore, any molecule capable of antagonizing the binding of the autoantibody to MOG protein in the myelin sheath will be useful for treating MS. Since MOGp is highly homologous to MOG, immunological cross-reactivity between these proteins is predicted. Therefore, soluble or solubilized derivatives of MOGp may disrupt the autoimmune response to MOG, and may be useful to treat or reduce the symptoms of MS. Suitable soluble derivatives of MOGp include the entire extracellular domain of MOGp or fragments thereof containing antigenic sites.

It has previously been shown that two immunodominant epitopes at the N-terminal of MOG, amino acids 1-125, are associated with the autoimmune response. Adelmann et al., J. Neuroimmunol. 63:17-27 (1995). Two immunodominant epitopes within this region are MOG₁₋₂₀ and MOG₃₅₋₅₅. Id. Due to the high degree of homology between MOGp and MOG within this region it is believed soluble fragments of MOGp comprising this N-terminal region would compete the binding of autoantibody for MOG or MOGp present in myelin or oligodendrocytes, ameliorating the autoimmune response. Therefore, the use of soluble peptides or polypeptides of MOGp comprising this N-terminal region, amino acids 1-125, especially residues 1-17 and 34-54, to treat MS or other demyelinating diseases represent preferred embodiments of this invention.

Modes of Administration

As a general proposition, the total pharmaceutically effective amount of soluble MOGp polypeptide administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the soluble MOGp polypeptide is typically administered at a dose rate of about 1 μg/kg/hour to about 50 μg/kg/hour, either by 1-4 injections per day or by continuous subcutaneous infusions, for example, using a mini-pump. An intravenous bag solution may also be employed.

Pharmaceutical compositions containing the soluble MOGp polypeptides of the invention may be administered orally, rectally, parenterally, intracistemally, intravaginally, intraperitoneally, topically (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrastemal, subcutaneous and intraarticular injection and infusion.

In addition to soluble MOGp polypeptides, MOGp polypeptides containing the transmembrane region can also be used when appropriately solubilized by including detergents with buffer. Detergents that can be used for solubilization include Triton, Tween, Chaps, Cholate, Deoxycholate, Brij, octylglucoside, and derivatives of these compounds.

Chromosome Assays

The nucleic acid molecules of the present invention are also valuable for chromosome identification. The sequence is specifically targeted to and can hybridize with a particular location on an individual human chromosome. The mapping of DNAs to chromosomes according to the present invention is an important first step in correlating those sequences with genes associated with disease.

In certain preferred embodiments in this regard, the cDNA herein disclosed is used to clone genomic DNA of a MOGp protein gene. This can be accomplished using a variety of well known techniques and libraries, which generally are available commercially. The genomic DNA then is used for in situ chromosome mapping using well known techniques for this purpose.

In addition, in some cases, sequences can be mapped to chromosomes by preparing PCR primers (preferably 15-25 bp) from the cDNA. Computer analysis of the 3′ untranslated region of the gene is used to rapidly select primers that do not span more than one exon in the genomic DNA, thus complicating the amplification process. These primers are then used for PCR screening of somatic cell hybrids containing individual human chromosomes.

Fluorescence in situ hybridization (“FISH”) of a cDNA clone to a metaphase chromosomal spread can be used to provide a precise chromosomal location in one step. This technique can be used with probes from the cDNA as short as 50 or 60 bp. For a review of this technique, see Verma et al., Human Chromosomes: a Manual Of Basic Techniques, Pergamon Press, New York (1988).

Once a sequence has been mapped to a precise chromosomal location, the physical position of the sequence on the chromosome can be correlated with genetic map data. Such data are found, for example, in V. McKusick, Mendelian Inheritance In Man, available on-line through Johns Hopkins University, Welch Medical Library. The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

The chromosomal position of the MOGp gene has been localized to 6p22-6p22.2.

Having generally described the invention, the same will be more readily understood by reference to the following examples, which are provided by way of illustration and are not intended as limiting.

### EXAMPLES

### Example 1

### Expression and Purification of the Extracellular Domain of MOGp in E. coli

The DNA sequence encoding the extracellular domain of MOGp protein in the deposited cDNA clone is amplified using PCR oligonucleotide primers specific to the amino terminal sequences of the MOGp protein and to sequences 3′ to the extracellular domain. Additional nucleotides containing restriction sites to facilitate cloning are added to the 5′ and 3′ sequences respectively.

The 5′ oligonucleotide primer has the sequence 5′ gga AGA TCT ctc ctt gct cag ctc agt ttt 3′ (SEQ ID NO:3) containing the underlined BglII restriction site, which encodes 21 nucleotides of the MOGp protein coding sequence in FIG. 1 (SEQ ID NO:1).

The 3′ primer has the sequence 5′ gcg cAG ATC Tct agg gct ggg cgc tcc tga aga a 3′ (SEQ ID NO:4) containing the underlined BglII restriction site followed by 24 nucleotides complementary to the 3′ coding sequence of immediately after the N-terminal extracellular domain.

For expression of the full-length protein the following 3′ primer is used: 5′ gga AGA TCT tta ttg gta tcg gac gga aga 3′ (SEQ ID NO:5)

The restriction sites are convenient to restriction enzyme sites in the bacterial expression vector pD10 (pQE9), which are used for bacterial expression in these examples. (Qiagen, Inc. 9259 Eton Avenue, Chatsworth, Calif., 91311). [pD10]pQE9 encodes ampicillin antibiotic resistance (“Ampr”) and contains a bacterial origin of replication (“ori”), an IPTG inducible promoter, a ribosome binding site (“RBS”), a 6-His tag and restriction enzyme sites.

The amplified MOGp DNA and the vector pQE9 both are digested with SalI and XbaI and the digested DNAs are then ligated together. Insertion of the MOGp extracellular domain DNA into the restricted pQE9 vector places the MOGp extracellular domain coding region downstream of and operably linked to the vector's IPTG-inducible promoter and in-frame with an initiating AUG appropriately positioned for translation of the MOGp extracellular domain.

The ligation mixture is transformed into competent E. coli cells using standard procedures. Such procedures are described in Sambrook et al., Molecular Cloning: A Laboratory Manual, 2nd Ed.; Cold Spring Harbor Laboratory Press, Cold Spring Harbor, N.Y. (1989). E. coli strain M15/rep4, containing multiple copies of the plasmid pREP4, which expresses lac repressor and confers kanamycin resistance (“Kan”), is used in carrying out the illustrative example described herein. This strain, which is only one of many that are suitable for expressing the MOGp extracellular domain or other MOGp polypeptides, is available commercially from Qiagen.

Transformants are identified by their ability to grow on LB plates in the presence of ampicillin and kanamycin. Plasmid DNA is isolated from resistant colonies and the identity of the cloned DNA confirmed by restriction analysis.

Clones containing the desired constructs are grown overnight (“O/N”) in liquid culture in LB media supplemented with both ampicillin (100 μg/ml) and kanamycin (25 μg/ml).

The O/N culture is used to inoculate a large culture, at a dilution of approximately 1:100 to 1:250. The cells are grown to an optical density at 600 nm (“OD600”) of between 0.4 and 0.6. Isopropyl-B-D-thiogalactopyranoside (“IPTG”) is then added to a final concentration of 1 mM to induce transcription from lac repressor sensitive promoters, by inactivating the lacI repressor. Cells subsequently are incubated further for 3 to 4 hours. Cells then are harvested by centrifugation and disrupted, by standard methods. Inclusion bodies are purified from the disrupted cells using routine collection techniques, and protein is solubilized from the inclusion bodies into 8M urea. The 8M urea solution containing the solubilized protein is passed over a PD-10 column in 2×phosphate-buffered saline (“PBS”), thereby removing the urea, exchanging the buffer and refolding the protein. The protein is purified by a further step of chromatography to remove endotoxin. Then, it is sterile filtered. The sterile filtered protein preparation is stored in 2×PBS at a concentration of 95 μg/ml.

### Example 2

### Expression and Purification of the MOGp Extracellular Domain Using the Baculovirus Expression System

The DNA sequence encoding the MOGp extracellular domain in the deposited cDNA clone is amplified using PCR oligonucleotide primers specific to the 5′ and 3′ sequence of the gene. Additional nucleotides containing restriction sites to facilitate cloning are added to the 5′ and 3′ sequences respectively.

The 5′ oligonucleotide primer has the sequence 5′ gcg cAG ATC Tcc gcc atc atg aaa atg gca agt tcc ctg 3′ (SEQ ID NO:6) (nucleotides 47 to 68) containing the underlined BglII restriction site followed by six nucleotides resembling an efficient translation initiation signal in eukaryotic cells, Kozak et al, J. Mol. Biol. 196:947-950 (1987), just behind the first 21 nucleotides of the human MOGp gene, with the ATG initiation codon underlined.

The 3′ primer has the sequence 5′ gcg cAG ATC Tct agg gct ggg cgc tcc tga aga a 3′ (SEQ ID NO:4) (nucleotides 765 to 786) containing the underlined BglII restriction site followed by 24 nucleotides complementary to the coding sequence immediately after the N-terminal extracellular domain of the MOGp gene.

The amplified fragment was isolated from a 1% agarose gel using a commercially available kit (“Geneclean,” BIO 101 Inc., La Jolla, Calif.). The fragment then was digested with BglII and again was purified on a 1% agarose gel. This fragment is designated herein F2.

The vector pA2, a modification of pVL 941 vector, was used to express the MOGp protein in the baculovirus expression system, using standard methods, as described in Summers et al., A Manual of Methods for Baculovirus Vectors and Insect Cell Culture Procedures, Texas Agricultural Experimental Station Bulletin No. 1555 (1987). This expression vector contains the strong polyhedrin promoter of the Autographa californica nuclear polyhedrosis virus (AcMNPV) followed by convenient restriction sites, e.g., BamHI and Asp 718. The polyadenylation site of the simian virus 40 (“SV40”) was used for efficient polyadenylation. For an easy selection of recombinant virus the beta-galactosidase gene from E. coli was inserted in the same orientation as the polyhedrin promoter and was followed by the polyadenylation signal of the polyhedrin gene. The polyhedrin sequences are flanked at both sides by viral sequences for cell-mediated homologous recombination with wild-type viral DNA to generate viable virus that express the cloned polynucleotide.

Many other baculovirus vectors could be used in place of pA2, such as pAc373, pVL941 and pAcIM1 provided, as those of skill readily will appreciate, that construction provides appropriately located signals for transcription, translation, trafficking and the like, such as an in-frame AUG and a signal peptide, as required. Such vectors are described in Luckow et al., Virology 170: 31-39, among others.

The plasmid was digested with the restriction enzyme BamHI and then was dephosphorylated using calf intestinal phosphatase, using routine procedures known in the art. The DNA was then isolated from a 1% agarose gel using a commercially available kit (“Geneclean” BIO 101 Inc., La Jolla, Calif.). This vector DNA is designated herein “V2”.

Fragment F2 and the dephosphorylated plasmid V2 were ligated together with T4 DNA ligase. E. coli HB101 cells were transformed and bacteria identified that contained the plasmid (pBAC MOGp) having the MOGp gene using the PCR method, in which one of the primers is that used to amplify the gene and the second primer in from well within the vector so that only those bacterial colonies containing the MOGp gene fragment will show amplification of the DNA. The sequence of the cloned fragment was confirmed by DNA sequencing. This plasmid is designated herein pBacMOGp.

5 μg of the plasmid pBacMOGp was co-transfected with 1.0 μg of a commercially available linearized baculovirus DNA (“BaculoGold™ baculovirus DNA”, Pharmingen, San Diego, Calif.), using the lipofection method described by Feigner et al., Proc. Natl. Acad. Sci. USA 84: 7413-7417 (1987). 1 μg of BaculoGold™ virus DNA and 5 μg of the plasmid pBacMOGp were mixed in a sterile well of a microtiter plate containing 50 μl of serum-free Grace's medium (Life Technologies Inc., Gaithersburg, Md.). Afterwards 10 μl Lipofectin plus 90 μl Grace's medium were added, mixed and incubated for 15 minutes at room temperature. Then the transfection mixture was added drop-wise to Sf9 (Spodoptera frugiperda) insect cells (ATCC CRL 1711) seeded in a 35 mm tissue culture plate with 1 ml Grace's medium without serum. The plate was rocked back and forth to mix the newly added solution. The plate was then incubated for 5 hours at 27° C. After 5 hours the transfection solution was removed from the plate and 1 ml of Grace's insect medium supplemented with 10% fetal calf serum was added. The plate was put back into an incubator and cultivation was continued at 27° C. for four days.

After four days the supernatant was collected and a plaque assay was performed, as described by Summers and Smith, cited above. An agarose gel with “Blue Gal” (Life Technologies Inc., Gaithersburg) was used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, Md., page 9-10).

Four days after serial dilution, the virus was added to the cells. After appropriate incubation, blue stained plaques were picked with the tip of a Pasteur pipette. The agar containing the recombinant viruses was then resuspended in an Eppendorf tube containing 200 μl of Grace's medium. The agar was removed by a brief centrifugation and the supernatant containing the recombinant baculovirus designated Baculovirus V-MOGp was used to infect Sf9 cells seeded in 35 mm dishes. Four days later the supernatants of these culture dishes were harvested and then they are stored at 4° C.

Sf9 cells were grown in EX-cell 401 medium (JRH) supplemented with 1% heat-inactivated FBS and 1% penicillin/streptomycin. The cells were infected with the recombinant baculovirus V-MOGp at a multiplicity of infection of 2. After 4 days, the medium was harvested through continuous centrifugation.

The supernatant was acidified to pH 4.5 with diluted acidic acid. After the resulting precipitation was removed by centrifugation, the medium was loaded onto a strong cation exchange column (Poros HS50, Perspective Biosystems) pre-equilibrated with 40 mM sodium acetate, pH 4.5. The column was washed with the same buffer and eluted with 0.5 M and 1.0 M NaCl. The desired protein was found in the flow through fractions as confirmed by SDS-PAGE analysis.

The pH of the flow through from the previous column was adjusted to 8.0 using NaOH. The media was then diluted 2-fold with water following the removal of precepitations through continuous centrifugation. The diluted sample was loaded again onto the strong anion exchange column (Poros HQ50, Perseptive Biosystem) equilibrated with 50 mM Tris-acetate, pH 8.5. Again, MOGp was found in the flow through fractions where the majority of contaminant were bound by the column.

The non-bound fractions (flow through) was again diluted 2-fold with water to reduce the conductivity of the solution below 5 ms. The diluted protein solution was then applied to a weak anion exchange column (Poros DEAE-50, Perseptive Biosystems) equilibrated with 25 mM Tris, pH 8.5. After washing the column using the same buffer, the column was eluted using a 30 column volume linear gradient ranging from 0 to 0.2M NaCl, 25 mM Tris, pH 8.5. Fractions were collected under constant A280 monitoring of the effluent and analyzed through SDS-PAGE. Those fractions containing MOGp (around 0.1M NaCl of the salt gradient) were then pooled.

The pooled fractions were adjusted to pH 7.0 using acidic acid followed by 5-fold dilution using water to keep conductivity of the solution below 2 ms. The sample was then loaded to a hydroxyaptite column (Bio-Rad) pre-equilibrated with 0.05×PBS. The column was eluted using 0.05×. 0.1× and 0.5×PBS. The fractions were analyzed using SDS-PAGE. The protein of interest was found in the fraction of 0.1×PBS elution of the column.

The resultant MOGp was of greater than 90% purity after the above purification steps which were carried out at 4-10° C.

This material was then electrophoresed in SDS polyacrylamide gels (Novex 4-20% gels) and transblotted onto a ProBlott membrane (Applied Biosystems, Inc. (ABI). After staining with Ponceau S (0.2% in 3% acetic acid), the band of interest was excised, and the single Asp/Pro bond was cleaved with formic acid at 37° C. for 3 days. The band was then placed in a “Blot Cartridge” and subjected to N-terminal amino acid sequence analysis using a model ABI-494 sequencer (Perkin-Elmer-Applied Biosystems, Inc.) and the Gas-phase Blot cycles. The underlined letters represent the N-terminal residues from the apparent mixture of components present in the sample and the colons indicate where the observed structures are identical to that expected from DNA sequencing.

Given below is the N-terminal sequence of MOGp obtained after the Asp-Pro cleavage of the protein, as described above:

| | |
|---|---|
| MKMASSLAFLLLNFGVSLLLVQLLTPCSAQFSVLGPSGPILAMVGEDADLPCHLFPTMSAETM | (SEQ ID NO:7) |
| ELKWVSSSLRQVVNVYADGKEVEDRQSAPYRGRTSILRDGITAGKAALRIHNVTASDSGKYLC | |
| YFQDGDFYEKALVELKVAALGSNLHVEVKGYEDGGIHLECRSTGWYPQPQIQWSNAKGENIPA | |
| VEAPVVADGVGLYEAVAASVIMRGGSGEGVSCII | |

p f f r s 60%

. . . . .

. . . . .

RNSLLGLEKTASISIADPFFRSAQPW* (SEQ ID NO:8)

The percentages refer to the approximate amount of material with the sequence shown.

### Example 3

### Cloning and Expression in Mammalian Cells

Most of the vectors used for the transient expression of the MOGp protein gene sequence in mammalian cells should carry the SV40 origin of replication. This allows the replication of the vector to high copy numbers in cells (e.g. COS cells) which express the T antigen required for the initiation of viral DNA synthesis. Any other mammalian cell line can also be utilized for this purpose.

A typical mammalian expression vector contains the promoter element, which mediates the initiation of transcription of mRNA, the protein coding sequence, and signals required for the termination of trancription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRs) from retroviruses, e.g. RSV, HTLV-1, HIV-1 and the early promoter of the cytomegalovirus (CMV). However, cellular signals can also be used (e.g. human actin promoter). Suitable expression vectors for use in practicing the present invention include, for example, vectors such as pSVL and pMSG (Pharmacia, Uppsala, Sweden), pRSVcat (ATCC 37152), pSV2dhfr (ATCC 37146) and pBC12MI (ATCC 67109). Mammalian host cells that could be used include, human HeLa, 283, H9 and Jurkart cells, mouse NIH3T3 and C127 cells, Cos 1, Cos 7 and CV1, African green monkey cells, quail QC1-3 cells, mouse L cells and Chinese hamster ovary cells.

Alternatively, the gene can be expressed in stable cell lines that contain the gene integrated into a chromosome. The co-transfection with a selectable marker such as dhfr, gpt, neomycin, hygromycin allows the identification and isolation of the transfected cells.

The transfected gene can also be amplified to express large amounts of the encoded protein. The DHFR (dihydrofolate reductase) is a useful marker to develop cell lines that carry several hundred or even several thousand copies of the gene of interest. Another useful selection marker is the enzyme glutamine synthase (GS) (Murphy et al., Biochem J. 227:277-279 (1991); Bebbington et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) cells are often used for the production of proteins.

The expression vectors pC1 and pC4 contain the strong promoter (LTR) of the Rous Sarcoma Virus (Cullen et al., Molecular and Cellular Biology, 438-4470 (March, 1985)) plus a fragment of the CMV-enhancer (Boshart et al., Cell 41:521-530 (1985)). Multiple cloning sites, e.g. with the restriction enzyme cleavage sites BamHI, XbaI and Asp718, facilitate the cloning of the gene of interest. The vectors contain in addition the 3′ intron, the polyadenylation and termination signal of the rat preproinsulin gene.

### Example 3(a)

### Cloning and Expression in COS Cells

The expression plasmid, pMOGp HA, was made by cloning a cDNA encoding MOGp into the expression vector pcDNA1/Amp (which can be obtained from Invitrogen, Inc.).

The expression vector pcDNA1/amp contains: (1) an E. coli origin of replication effective for propagation in E. coli and other prokaryotic cells; (2) an ampicillin resistance gene for selection of plasmid-containing prokaryotic cells; (3) an SV40 origin of replication for propagation in eukaryotic cells; (4) a CMV promoter, a polylinker, an SV40 intron, and a polyadenylation signal arranged so that a cDNA conveniently can be placed under expression control of the CMV promoter and operably linked to the SV40 intron and the polyadenylation signal by means of restriction sites in the polylinker.

A DNA fragment encoding the MOGp protein and an HA tag fused in frame to its 3′ end was cloned into the polylinker region of the vector so that recombinant protein expression was directed by the CMV promoter. The HA tag corresponds to an epitope derived from the influenza hemagglutinin protein described by Wilson et al., Cell 37: 767 (1984). The fusion of the HA tag to the target protein allows easy detection of the recombinant protein with an antibody that recognizes the HA epitope.

The plasmid construction strategy was as follows. The MOGp cDNA of the deposited clone was amplified using primers that contain convenient restriction sites, as described above regarding the construction of expression vectors for expression of MOGp in E. coli. To facilitate detection, purification and characterization of the expressed MOGp, one of the primers contains a hemagglutinin tag (“HA tag”) as described above.

Suitable primers include the following, which are used in this example. The 5′ primer, containing the underlined BglII site, an AUG start codon and 7 codons of the 5′ coding region has the following sequence:

5′gcg cAG ATC Tcc gcc atc atg aaa atg gca agt tcc ctg 3′ (SEQ ID NO:6).

The 3′ primer, containing complementary sequence to the underlined BglII site, a stop codon, 9 codons thereafter forming the hemagglutinin HA tag, and 23 bp of 3′ coding sequence after the N-terminal extracellular domain, not including the stop codon (at the 3′ end) has the following sequence:

5′ gcg cAG ATC Tct agg gct ggg cgc tcc tga aga a 3′ (SEQ ID NO:4).

The PCR amplified product contains a BglII site, 23 nucleotides of the human MOGp coding sequence, followed by the HA fused in frame, a translation termination stop codon next to the HA tag, and a BglII site.

The PCR amplified DNA fragment was digested with BglII and the vector, pcDI/Amp, was digested with BamHI and then fragments were ligated. The ligation mixture was transformed into E. coli strain SURE (available from Stratagene Cloning Systems, 11099 North Torrey Pines Road, La Jolla, Calif. 92037), and the transformed culture was plated on ampicillin media plates which then were incubated to allow growth of ampicillin resistant colonies. Plasmid DNA was isolated from resistant colonies and examined by restriction analysis and gel sizing for the presence of the MOGp-encoding fragment.

For expression of recombinant MOGp, COS cells were transfected with an expression vector, as described above, using DEAE-DEXTRAN, as described, for instance, in Sambrook et al., Molecular Cloning: A Laboratory Manual, Cold Spring Laboratory Press, Cold Spring Harbor, N.Y. (1989). Cells were incubated under conditions for expression of MOGp by the vector.

Expression of the MOGp-HA fusion protein was detected by radiolabelling and immunoprecipitation, using methods described in, for example Harlow et al., Antibodies: A Laboratory Manual, 2nd Ed.; Cold Spring Harbor Laboratory Press, Cold Spring Harbor, N.Y. (1988). To this end, two days after transfection, the cells were labeled by incubation in media containing ³⁵S-cysteine for 8 hours. The cells and the media were collected, and the cells were washed and then lysed with detergent-containing RIPA buffer: 150 mM NaCl, 1% NP-40, 0.1% SDS, 1% NP-40, 0.5% DOC, 50 mM TRIS, pH 7.5, as described by Wilson et al. cited above. Proteins were precipitated from the cell lysate and from the culture media using an HA-specific monoclonal antibody. The precipitated proteins then were analyzed by SDS-PAGE gels and autoradiography. An expression product of the expected size is seen in the cell lysate, which is not seen in negative controls.

### Example 3(b)

### Cloning and Expression in CHO Cells

The vector pC1 is used for the expression of MOGp protein. Plasmid pC1 is a derivative of the plasmid pSV2-dhfr [ATCC Accession No. 37146]. Both plasmids contain the mouse DHFR gene under control of the SV40 early promoter. Chinese hamster ovary- or other cells lacking dihydrofolate activity that are transfected with these plasmids can be selected by growing the cells in a selective medium (alpha minus MEM, Life Technologies) supplemented with the chemotherapeutic agent methotrexate. The amplification of the DHFR genes in cells resistant to methotrexate (MTX) has been well documented (see, e.g., Alt, F. W., et al., J. Biol. Chem. 253:1357-1370 (1978); Hamlin, J. L. and Ma, C., Biochem. et Biophys. Acta, 1097:107-143; Page, M. J. and Sydenham, M. A. 1991, Biotechnology 9:64-68 (1991)). Cells grown in increasing concentrations of MTX develop resistance to the drug by overproducing the target enzyme, DHFR, as a result of amplification of the DHFR gene. If a second gene is linked to the DHFR gene it is usually co-amplified and over-expressed. It is state of the art to develop cell lines carrying more than 1,000 copies of the genes. Subsequently, when the methotrexate is withdrawn, cell lines contain the amplified gene integrated into the chromosome(s).

Plasmid pC1 contains for the expression of the gene of interest a strong promoter of the long terminal repeat (LTR) of the Rouse Sarcoma Virus (Cullen, et al., Molec. Cell Biol, 5(3):438-4470 (1985)) plus a fragment isolated from the enhancer of the immediate early gene of human cytomegalovirus (CMV) (Boshart et al., Cell 41:521-530 (1985)). Downstream of the promoter is a BamHI restriction enzyme cleavage site that allows the integration of the genes followed by the 3′ intron and the polyadenylation site of the rat preproinsulin gene. Other high efficient promoters can also be used for the expression, e.g., the human β-actin promoter, the SV40 early or late promoters, or the long terminal repeats from other retroviruses, e.g., HIV and HTLV-1. For the polyadenylation of the mRNA other signals, e.g., from the human growth hormone or globin genes can be used as well.

Stable cell lines carrying a gene of interest integrated into the chromosomes can also be selected upon co-transfection with a selectable marker such as gpt, G418 or hygromycin. It is advantageous to use more than one selectable marker in the beginning, e.g. G418 plus methotrexate.

The plasmid pC1 is digested with the restriction enzyme BamHI and then dephosphorylated using calf intestinal phosphates by procedures known in the art. The vector is then isolated from a 1% agarose gel.

The DNA sequence encoding MOGp, ATCC No. 97709, is amplified using PCR oligonucleotide primers corresponding to the 5′ and 3′ sequences of the gene:

The 5′ primer has the sequence 5′ gcg cAG ATC Tcc gcc atc atg aaa atg gca agt tcc ctg 3′ (SEQ ID NO:6) containing the underlined BglII restriction enzyme site followed by 6 bases resembling an efficient eukoryotic translation initiation signal, followed by 21 bases of the sequence of MOGp of FIG. 1 (SEQ ID NO:1). Inserted into an expression vector, as described below, the 5′ end of the amplified fragment encoding human MOGp provides an efficient signal peptide. An efficient signal for initiation of translation in eukaryotic cells, as described by Kozak, M., J. Mol. Biol. 196:947-950 (1987) is appropriately located in the vector portion of the construct.

The 3′ primer has the sequence 5′ gcg cAG ATC Tct agg gct ggg cgc tcc tga aga a 3′ (SEQ ID NO:4) containing the underlined BglII restriction site followed by 24 nucleotides, including the stop codon, complementary to the 24 coding sequences immediately after the N-terminal extracellular domain.

For expression of the full-length protein the following 3′ primer is used: 5′ gga AGA TCT tta ttg gta tcg gac gga aga 3′ (SEQ ID NO:5).

The amplified fragments are isolated from a 1% agarose gel as described above and then digested with the endonuclease BamHI and then purified again on a 1% agarose gel.

The isolated fragment and the dephosphorylated vector are then ligated with T4 DNA ligase. E. coli HB101 cells are then transformed and bacteria identified that contained the plasmid pC1 inserted in the correct orientation using the restriction enzyme BamHI. The sequence of the inserted gene is confirmed by DNA sequencing.

Transfection of CHO-DHFR-Cells

Chinese hamster ovary cells lacking an active DHFR enzyme are used for transfection. 5 μg of the expression plasmid C1 are cotransfected with 0.5 μg of the plasmid pSVneo using the lipofecting method (Felgner et al., supra). The plasmid pSV2-neo contains a dominant selectable marker, the gene neo from Tn5 encoding an enzyme that confers resistance to a group of antibiotics including G418. The cells are seeded in alpha minus MEM supplemented with 10, 25, or 50 ng/ml of methotrexate plus 1 mg/ml G418. After 2 days, the cells are trypsinized and seeded in hybridoma cloning plates (Greiner, Germany) and cultivated for 10-14 days. After this period, single clones are trypsinized and then seeded in 6-well petri dishes using different concentrations of methotrexate (25 nM, 50 nM, 100 nM, 200 nM, 400 nM). Clones growing at the highest concentrations of methotrexate are then transferred to new 6-well plates containing even higher concentrations of methotrexate (500 nM, 1 μM, 2 μM, 5 μM). The same procedure is repeated until clones grow at a concentration of 100 μM.

The expression of the desired gene product is analyzed by Western blot analysis and SDS-PAGE.

### Example 4

### Tissue Distribution of MOGp Protein Expression

Northern blot analysis is carried out to examine MOGp gene expression in human tissues, using methods described by, among others, Sambrook et al., cited above. A cDNA probe containing the entire nucleotide sequence of the MOGp protein (SEQ ID NO:1) is labeled with 12p using the Rediprime™ DNA labeling system (Amersham Life Science), according to manufacturer's instructions. After labeling, the probe is purified using a CHROMA SPIN-100™ column (Clontech Laboratories, Inc.), according to manufacturer's protocol number PT1200-1. The purified labeled probe is then used to examine various human tissues for MOGp mRNA.

Multiple Tissue Northern (MTN) blots containing various human tissues (H) or human immune system tissues (IM) are obtained from Clontech and are examined with labeled probe using ExpressHyb™ hybridization solution (Clontech) according to manufacturer's protocol number PT 1190-1. Following hybridization and washing, the blots are mounted and exposed to film at −70° C. overnight, and films developed according to standard procedures. From Northern blot analysis expression of this gene was detected in peripheral blood lymphocytes, spleen, and bone marrrow. Tissues in which expression of this gene was not detected are pancreas, kidney, muscle, liver, lung, placenta, brain and heart. This suggests that MOGp is probably involved in lymphocyte function such as lymphopoises, lymphocyte homing and activation, hematopoises, tumor progression, and metastasis. Since MOGp is also an immunoglobulin-like molecule, it is quite likely that MOGp is also involved in immune system signaling and/or immune interactions.

It will be clear that the invention may be practiced otherwise than as particularly described in the foregoing description and examples.

Numerous modifications and variations of the present invention are possible in light of the above teachings and, therefore, are within the scope of the appended claims.

The entire disclosure of all publications (including patents, patent applications, journal articles, laboratory manuals, books, or other documents) cited herein are hereby incorporated by reference.

What is claimed is:

1. An isolated nucleic acid molecule comprising a polynucleotide having a nucleotide sequence at least 95% identical to a sequence selected from the group consisting of:
(a) a nucleotide sequence encoding the MOGp polypeptide having the complete amino acid sequence in FIG. 1 (SEQ ID NO:2);
(b) a nucleotide sequence encoding the MOGp polypeptide having the amino acid sequence at positions 2-331 in FIG. 1 (SEQ ID NO:2);
(c) a nucleotide sequence encoding the mature MOGp polypeptide having the amino acid sequence at positions 30-331 in FIG. 1 (SEQ ID NO:2);
(d) a nucleotide sequence encoding the MOGp polypeptide having the complete amino acid sequence encoded by the cDNA clone contained in ATCC Deposit No. 97709;
(e) a nucleotide sequence encoding the mature MOGp polypeptide having the amino acid sequence encoded by the cDNA clone contained in ATCC Deposit No. 97709;
(f) a nucleotide sequence encoding the MOGp polypeptide extracellular domain having an amino acid sequence at positions 30 to 247 shown in FIG. 1 [SEQ ID NO:2];
(g) a nucleotide sequence encoding the MOGp polypeptide transmembrane domain having an amino acid sequence at positions 248 to 271 shown in FIG. 1 [SEQ ID NO:2];
(h) a nucleotide sequence encoding the MOGp polypeptide intracellular domain having an amino acid sequence at positions 272 to 331 shown in FIG. 1 [SEQ ID NO:2];
(i) a nucleotide sequence encoding the MOGp receptor extracellular and intracellular domains with all or part of the transmembrane domain deleted; and
(j) a nucleotide sequence complementary to any of the nucleotide sequences in (a), (b), (c), (d), (e), (f), (g), (h), or (i).

2. The nucleic acid molecule of claim 1, wherein said polynucleotide has the complete nucleotide sequence in FIG. 1 (SEQ ID NO:1).

3. The nucleic acid molecule of claim 1 wherein said polynucleotide has the nucleotide sequence in FIG. 1 (SEQ ID NO:1) encoding the MOGp polypeptide having the complete amino acid sequence in FIG. 1 (SEQ ID NO:2).

4. The nucleic acid molecule of claim 1 wherein said polynucleotide has the nucleotide sequence in FIG. 1 (SEQ ID NO:1) encoding the mature polypeptide having the amino acid sequence at positions 30-331 in FIG. 1 (SEQ ID NO:2).

5. The nucleic acid molecule of claim 1, wherein said polynucleotide has the complete nucleotide sequence of the cDNA clone contained in ATCC Deposit No. 97709.

6. The nucleic acid molecule of claim 1, wherein said polynucleotide has the nucleotide sequence encoding the MOGp polypeptide having the complete amino acid sequence encoded by the cDNA clone contained in ATCC Deposit No. 97709.

7. The nucleic acid molecule of claim 1, wherein said polynucleotide has the nucleotide sequence encoding the mature MOGp polypeptide having the amino acid sequence encoded by the cDNA clone contained in ATCC Deposit No. 97709.

8. The nucleic acid molecule of claim 1, wherein said polynucleotide has a nucleotide sequence at least 95% identical to a sequence encoding MOGp extracellular domain and intracellular domain, wherein all or a part of the transmembrane domain is deleted.

9. An isolated nucleic acid molecule comprising a polynucleotide which hybridizes under stringent hybridization conditions to a polynucleotide having a nucleotide sequence identical to a nucleotide sequence in (a), (b), (c), (d), (e), (f), (g) or (h) of claim 1 wherein said polynucleotide which hybridizes does not hybridize under stringent hybridization conditions to a polynucleotide having a nucleotide sequence consisting of only A residues or of only T residues.

10. An isolated nucleic acid molecule comprising a polynucleotide which encodes the amino acid sequence of an epitope-bearing portion of a MOGp polypeptide having an amino acid sequence in (a), (b), (c), (d), (e), (f), (g) or (h) of claim 1.

11. The isolated nucleic acid molecule of claim 10, which encodes an epitope-bearing portion of a MOGp polypeptide selected from the group consisting of: a polypeptide comprising amino acid residues from about 1 to about 125 in FIG. 1 (SEQ ID NO:2); a polypeptide comprising amino acid residues from about 1 to about 55 in FIG. 1 (SEQ ID NO:2); a polypeptide comprising amino acid residues from about 80 to about 113 in FIG. 1 (SEQ ID No:2); a polypeptide comprising amino acid residues from about 282 to about 297 in FIG. 1 (SEQ ID No:2); a polypeptide comprising amino acid residues from about 299 to about 331 in FIG. 1 (SEQ ID No:2); a polypeptide comprising amino acid residues from about 46 to about 53 in FIG. 1 (SEQ ID No:2); a polypeptide comprising amino acid residues from about 59 to about 65 in FIG. 1 (SEQ ID No:2); a polypeptide comprising amino acid residues from about 71 to about 77 in FIG. 1 (SEQ ID No:2); a polypeptide comprising amino acid residues from about 119 to about 125 in FIG. 1 (SEQ ID No:2); a polypeptide comprising amino acid residues from about 130 to about 137 in FIG. 1 (SEQ ID No:2); a polypeptide comprising amino acid residues from about 183 to about 190 in FIG. 1 (SEQ ID No:2); a polypeptide comprising amino acid residues from about 211 to about 219 in FIG. 1 (SEQ ID No:2); a polypeptide comprising amino acid residues from about 239 to about 248 in FIG. 1 (SEQ ID No:2); and a polypeptide comprising amino acid residues from about 275 to about 280 in FIG. 1 (SEQ ID No:2).

12. The isolated nucleic acid molecule of claim 1, which encodes a soluble polypeptide comprising the MOGp receptor extracellular domain.

13. The isolated nucleic acid molecule of claim 1, which encodes the MOGp receptor transmembrane domain.

14. The isolated nucleic acid molecule of claim 1, which encodes a soluble polypeptide comprising the MOGp receptor intracellular domain.

15. An isolated nucleic acid molecule comprising a polynucleotide having a sequence at least 95% identical to a sequence selected from the group consisting of:
(a) the nucleotide sequence of a fragment of the sequence shown in SEQ ID NO:1, wherein said fragment comprises at least 50 contiguous nucleotides of SEQ ID NO:1, provided that said nucleotide sequence is not HAFAV34R (SEQ ID NO. 11); HETBC89R (SEQ ID. NO. 12); HRDDL76R (SEQ ID. NO. 13); HRDDL35R (SEQ ID NO. 14); HRDDI47R (SEQ ID NO. 15); HRDDK16R (SEQ ID NO. 16); HRDDK03R (SEQ ID NO. 17); HRDDK54R (SEQ ID NO. 18); HRDBQ91R (SEQ ID NO. 19); HRDCB31R (SEQ ID NO. 20); HRDDL95R (SEQ ID NO. 21); HFCAE49F (SEQ ID NO. 22); HTWAL13R (SEQ ID NO. 23); T91685 (SEQ ID NO. 24); AA303854 (SEQ ID NO. 25); T70127 (SEQ ID NO. 26); T86577 (SEQ ID NO. 27); AA337675 (SEQ ID NO. 28); T94934 (SEQ ID NO. 29); AA114263 (SEQ ID NO. 30); T92875 (SEQ ID NO. 31); AA484820 (SEQ ID NO. 32); T70246 (SEQ ID NO. 33); AA134341 (SEQ ID NO. 34); AA134342 (SEQ ID NO. 35); T94480 (SEQ ID NO. 36); T89056 (SEQ ID NO. 37); T86754 (SEQ ID NO. 38); and T98146 (SEQ ID NO. 39) or any subfragment thereof; and
(b) a nucleotide sequence complementary to a nucleotide sequence in (a).

16. A method for making a recombinant vector comprising inserting an isolated nucleic acid molecule of claim 1 into a vector.

17. A recombinant vector produced by the method of claim 16.

18. A method of making a recombinant host cell comprising introducing the recombinant vector of claim 17 into a host cell.

19. A recombinant host cell produced by the method of claim 18.

20. A recombinant method for producing a MOGp polypeptide, comprising culturing the recombinant host cell of claim 18 under conditions such that said polypeptide is expressed and recovering said polypeptide.

21. An isolated MOGp polypeptide having an amino acid sequence at least 95% identical to a sequence selected from the group consisting of:
(a) the amino acid sequence of the MOGp polypeptide having the complete amino acid sequence in FIG. 1 (SEQ ID NO:2);
(b) the amino acid sequence of the MOGp polypeptide having the amino acid sequence at positions 2-331 in FIG. 1 (SEQ ID NO:2);
(c) the amino acid sequence of the mature MOGp polypeptide having the amino acid sequence at positions 30-331 in FIG. 1 (SEQ ID NO:2);
(d) the amino acid sequence of the MOGp polypeptide having the complete amino acid sequence encoded by the cDNA clone contained in ATCC Deposit No. 97709;
(e) the amino acid sequence of the mature MOGp polypeptide having the amino acid sequence encoded by the cDNA clone contained in ATCC Deposit No. 97709; and
(f) the amino acid sequence of the MOGp extracellular domain having an amino acid sequence at positions 30 to 250 shown in FIG. 1;
(g) the amino acid sequence of the MOGp transmembrane domain having an amino acid sequence at positions 251 to 270 shown in FIG. 1;
(h) the amino acid sequence of the MOGp intracellular domain having an amino acid sequence at positions 271 to 331 shown in FIG. 1; and
(i) the amino acid sequence of an epitope-bearing portion of any one of the polypeptides of (a), (b), (c), (d), (e), (f), (g), or (h).

22. An isolated polypeptide comprising an epitope-bearing portion of the MOGp protein, wherein said portion is selected from the group consisting of: a polypeptide comprising amino acid residues from about 1 to about 125 in FIG. 1 (SEQ ID NO:2); a polypeptide comprising amino acid residues from about 1 to about 55 in FIG. 1 (SEQ ID NO:2); a polypeptide comprising amino acid residues from about 1 to about 55 in FIG. 1 (SEQ ID NO:2); a polypeptide comprising amino acid residues from about 80 to about 113 in FIG. 1 (SEQ ID No:2); a polypeptide comprising amino acid residues from about 282 to about 297 in FIG. 1 (SEQ ID No:2); a polypeptide comprising amino acid residues from about 299 to about 331 in FIG. 1 (SEQ ID No:2); a polypeptide comprising amino acid residues from about 46 to about 53 in FIG. 1 (SEQ ID No:2); a polypeptide comprising amino acid residues from about 59 to about 65 in FIG. 1 (SEQ ID No:2); a polypeptide comprising amino acid residues from about 71 to about 77 in FIG. 1 (SEQ ID No:2); a polypeptide comprising amino acid residues from about 119 to about 125 in FIG. 1 (SEQ ID No:2); a polypeptide comprising amino acid residues from about 130 to about 137 in FIG. 1 (SEQ ID No:2); a polypeptide comprising amino acid residues from about 183 to about 190 in FIG. 1 (SEQ ID No:2); a polypeptide comprising amino acid residues from about 211 to about 219 in FIG. 1 (SEQ ID No:2); a polypeptide comprising amino acid residues from about 239 to about 248 in FIG. 1 (SEQ ID No:2); and a polypeptide comprising amino acid residues from about 275 to about 280 in FIG. 1 (SEQ ID No:2).

23. An isolated antibody that binds specifically to a MOGp polypeptide of claim 21.

24. A method of diagnosing multiple sclerosis comprising:
(a) providing a biological sample from an individual to be tested for multiple sclerosis;
(b) assaying said biological sample for the amount of antibody to MOGp protein present in said biological sample;
(c) comparing the amount of antibody to MOGp protein in said biological sample to the amount of antibody to MOGp protein in a standard sample from an individual not having multiple sclerosis; and
(d) correlating an enhanced amount of the antibody in said biological sample relative to said standard with an increased probability of multiple sclerosis.

25. The method of diagnosing multiple sclerosis as claimed in claim 24, wherein said biological sample is sera or plasma from a human suspected of having multiple sclerosis.

26. A method used for the diagnosis of a tumor or inflammatory disease, comprising:
(a) assaying MOGp protein gene expression level in mammalian cells or body fluid; and
(b) comparing said MOGp protein gene expression level with a standard MOGp protein gene expression level whereby an increase in said MOGp gene expression level over said standard is indicative of an increased probability of a tumor or inflammatory disease.

27. The method of claim 26, wherein said MOGp gene expression level is assayed by detecting MOGp protein with an antibody.

28. The method of claim 26, wherein said MOGp gene expression level is assayed by detecting MOGp mRNA levels.

29. The isolated nucleic acid molecule as claimed in claim 1, wherein said isolated nucleic acid molecule is not the nucleic acid molecule or nucleic acid insert identified in the following GenBank Accession Reports: T91685, AA303854, T70127, T86577, AA337675, T94934, AA114263, T92875, AA484820, T70246, AA134341, T94480, T89056, T86754, and T98146.

30. An isolated nucleic acid molecule comprising a MOGp structural gene operably linked to a heterologous promoter.

31. The isolated nucleic acid molecule as claimed in claim 30, wherein said isolated nucleic acid molecule does not encode a fusion protein comprising the MOGp structural gene or a fragment thereof.

32. The isolated nucleic acid molecule as claimed in claim 30, wherein said isolated nucleic acid molecule does not encode a β-galactosidease-MOGp fusion protein.

33. The isolated nucleic acid molecule as claimed in claim 30, wherein said isolated nucleic acid molecule is capable of expressing a MOGp polypeptide, wherein said MOGp polypeptide does not contain and is not covalently linked to an amino acid sequence encoded by the 5′ untranslated portion of the MOGp gene.

34. The isolated nucleic acid molecule as claimed in claim 1, wherein said isolated nucleic acid does not contain a nucleotide sequence at least 90% identical or 90% complementary to the 3′ untranslated region of FIG. 1 or a fragment thereof greater than 25 nucleotides in length.

35. The isolated nucleic acid molecule as claimed in claim 1, wherein said isolated nucleic acid does not contain a nucleotide sequence at least 90% identical or 90% complementary to the 5′ untranslated region of FIG. 1 or a fragment thereof greater than 25 nucleotides in length.
